(19) United States
(12) Patent Application Publication
(10) Pub. No.: US 2008/0090836 A1
(43) Pub. Date: Apr. 17, 2008

(54) Pyrazole Compounds Useful In The Treatment Of Inflammation

(76) Inventors: Peter Nilsson, Solna (SE); Andrei Sanin, Solna (SE); Benjamin Pelcman, Solna (SE); Thomas Boesen, Copenhagen (DK)

Correspondence Address: MORGAN LEWIS & BOCKIUS LLP, 1111 PENNSYLVANIA AVENUE NW, WASHINGTON, DC, 20004, US

(21) Appl. No.: 11/663,180

(22) PCT Filed: Sep. 19, 2005
(86) PCT No.: PCT/GB05/03584

§ 371 Date: Mar. 19, 2007

Related U.S. Application Data
(60) Provisional application No. 60/610,952, filed on Sep. 20, 2004.

(51) Int. Cl. A61K 31/415; A61K 31/454; A61K 31/496; A61P 29/00; C07D 207/34; C07D 401/02; C07D 403/02

(52) U.S. Cl. 514/254.05; 514/326; 514/406; 544/371; 546/211; 548/374.1

(57) ABSTRACT

There is provided compounds of formula (I), wherein R^{a}, R^{b}, R¹, R², R³, X and Y have meanings given in the description, and pharmaceutically-acceptable salts thereof, which compounds are useful in the treatment of diseases in which inhibition of the activity of a lipoxygenase (e.g. 15-lipoxygenase) is desired and/or required, and particularly in the treatment of inflammation.

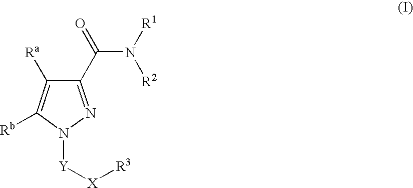

### FIELD OF THE INVENTION

The invention relates to novel pharmaceutically-useful compounds. The invention further relates to compounds that are useful in the inhibition of the activity of 15-lipoxygenase and thus in the treatment of inflammatory diseases and of inflammation generally. The invention also relates to the use of such compounds as medicaments, to pharmaceutical compositions containing them, and to synthetic routes for their production.

### BACKGROUND

There are many diseases/disorders that are inflammatory in their nature. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, leukotriene receptor antagonists (LTRas) have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, a new and/or alternative anti-inflammatory treatment would be of benefit to all of the above-mentioned patient groups. In particular, there is a real and substantial unmet clinical need for an effective anti-inflammatory drug capable of treating inflammatory disorders, such as asthma, with no real or perceived side effects.

The mammalian lipoxygenases are a family of structurally-related enzymes, which catalyze the oxygenation of arachidonic acid. Three types of human lipoxygenases are known, which catalyze the insertion of molecular oxygen into arachidonic acid at carbon positions 5, 12 and 15. The enzymes are thus named 5-, 12- and 15-lipoxygenase, respectively.

Arachidonic acid metabolites that are formed following the action of lipoxygenases are known to have pronounced pathophysiological activity including pro-inflammatory effects.

For example, the primary product of the action of 5-lipoxygenase on arachidonic acid is further converted by a number of enzymes to a variety of physiologically and pathophysiologically important metabolites. The most important of these, the leukotrienes, are strong bronchoconstrictors. Huge efforts have been devoted towards the development of drugs that inhibit the action of these metabolites as well as the biological processes that form them. Drugs that have been developed to this end include 5-lipoxygenase inhibitors, inhibitors of FLAP (Five Lipoxygenase Activating Protein) and, as mentioned previously, leukotriene receptor antagonists (LTRas).

Another class of enzymes that metabolize arachidonic acid are the cyclooxygenases. Arachidonic acid metabolites that are produced by this process include prostaglandins, thromboxanes and prostacyclin, all of which possess physiological or pathophysiological activity. In particular, the prostaglandin PGE₂ is a strong pro-inflammatory mediator, which also induces fever and pain. Consequently, a number of drugs have been developed to inhibit the formation of PGE₂, including “NSAIDs” (non-steroidal antiinflammatory drugs) and “coxibs” (selective cyclooxygenase-2 inhibitors). These classes of compounds act predominantly by way of inhibition of one or several cyclooxygenases.

Thus, in general, agents that are capable of blocking the formation of arachidonic acid metabolites are likely to be of benefit in the treatment of inflammation.

### PRIOR ART

Certain pyrazolecarboxylic acid hydrazides, which are structurally unrelated to the compounds described herein, have been disclosed as anti-inflammatory agents in Tihanyi et al, Eur. J. Med. Chem.—Chim. Ther., 1984, 19, 433 and Goel et al, J. Chem. Inf. Comput. Sci. 1995, 35, 510.

Heterocyclic compounds (including pyrazoles, such as 1-acetyl-3-(2,6-dimethylphenylcarbamoyl)-5-methylpyrazole) with anticonvulsant activity have been disclosed in inter alia U.S. Pat. No. 5,258,397 and U.S. Pat. No. 5,464,860. Other heterocyclic compounds, including pyrazoles, have been disclosed for use as Factor Xa inhibitors in international patent applications WO 01/19788 and WO 02/00651 and for use as cannabinoid receptors in international patent application WO 01/58869. None of these documents disclose or suggest the use of the compounds disclosed therein in the treatment of inflammation and/or as inhibitors of lipoxygenases.

International patent application WO 99/25695 discloses various pyrazole compounds for use in the treatment of inflammation. However, this document does not disclose or suggest pyrazoles that are substituted at the pyrazole nitrogen by a carbonyl, a thiocarbonyl or a sulfonyl group.

International application WO 03/037274 discloses various pyrazoles that may be useful in treating inflammatory pain, which mechanism works by blocking sodium channels. International application WO 03/068767 also discloses inter alia pyrazole-containing compounds that may be useful in treating inflammatory pain by opening potassium ion channels. However, there is no specific disclosure in either of these documents of 3-amido pyrazoles that have a linker group at the 1(N)-position.

International patent application WO 2004/080999 discloses 1-substituted pyrazole derivatives for use in the treatment of inflammation. Compounds comprising substituents at the 4- and/or 5-positions of the pyrazole unit are neither mentioned nor suggested in this document.

International patent application WO 2004/056815 discloses various pyrazoles that may be useful as Factor Xa inhibitors. There is no specific disclosure in this document of 3-amido-1(N)-substituted pyrazoles, in which the NT-substituent is attached via a carbonyl, a thiocarbonyl or a sulfonyl group.

International patent application WO 96/11917 discloses various compounds that may be useful in treating inflammation. This document only discloses benzoxazoles and benzothiazoles substituted, directly or via an alkyl linker group, in the 2-position by an aromatic group.

Finally, Vertuani et al., Journal of Pharmaceutical Sciences, Vol. 74, No. 9 (1985) discloses various pyrazoles that possess anti-inflammatory and analgesic activities. There is no mention or suggestion of 1(N)-substituted pyrazoles.

### DISCLOSURE OF THE INVENTION

According to the invention there is provided a compound of formula I, 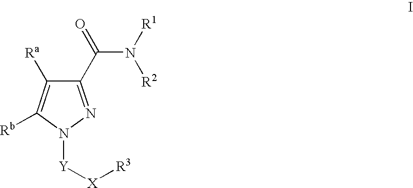
wherein
either
R¹ represents an aryl group or a heteroaryl group, both of which are optionally substituted by one or more substituents selected from G¹ and B¹, which B¹ group may itself be further substituted by one or more substituents selected from G², Z (wherein Z is not directly attached to an aryl or a heteroaryl group) and B² (which B² group is optionally further substituted by one or more substituents selected from G³, B³ and Z, wherein Z is not attached to an aryl or a heteroaryl group); and R² represents H or C₁₋₈ alkyl, which latter group is optionally substituted by one or more halo groups;
or
when R² represents C₁₋₈ alkyl optionally substituted by halo, R¹ and R² may be linked together forming a further 5- to 7-membered ring, optionally containing 1 to 3 heteroatoms and/or 1 to 3 double bonds, which ring is itself optionally substituted by one or more substituents selected from G¹, Z (when the ring is not aromatic in nature) and B¹ (which B¹ group is optionally substituted as described above);
R³ represents C₁₋₈ alkyl, heterocycloalkyl, aryl or heteroaryl, all of which groups are optionally substituted by one or more substituents selected from G¹, Z (when Z is not directly attached to an aryl or a heteroaryl group) and B₁ (which B¹ group is optionally substituted as described above);
X represents a direct bond or —N(R^{4a})—;
Y represents —C(O)—, —C(S)— or —S(O)₂—;
B¹, B² and B³ independently represent, on each occasion when used above, C₁₋₈ allyl, heterocycloalkyl, aryl or heteroaryl;
G¹, G² and G³ independently represent, on each occasion when used above, halo, cyano, —N₃, —NO₂, —ONO₂ or -A¹-R^{4b};
wherein A¹ represents a spacer group selected from —C(Z)A²-, —N(R⁵)A³-, —OA⁴-, —S— or —S(O)ₙA⁵-, in which:
A² represents a single bond, —O—, —S— or —N(R⁵)—;
A³ represents A⁶, —C(Z)N(R⁵)C(Z)N(R⁵)—, —C(Z)N(R⁵)C(Z)O—, —C(Z)N(R⁵)S(O)ₙN(R⁵)—, —C(Z)S—, —S(O)ₙ—, —S(O)ₙN(R⁵)C(Z)N(R⁵)—, —S(O)ₙN(R⁵)C(Z)O—, —S(O)ₙN(R⁵)S(O)ₙN(R⁵)—, —C(Z)O—, —S(O)ₙN(R⁵)— or —S(O)ₙO—;
A⁴ represents A⁶, —S(O)ₙ—, —C(Z)O—, —S(O)ₙN(R⁵)— or —S(O)ₙO—;
A⁵ represents a single bond, —N(R⁵)— or —O—;
A⁶ represents a single bond, —C(Z)- or —C(Z)N(R⁵)—;

Z represents, on each occasion when used above, a substituent connected by a double bond, which is selected from ═O, ═S, ═N^{R4b}, ═NN(R^{4b})(R⁵), ═NOR^{4b}, ═NS(O)₂N(R^{4b})(R⁵), ═NCN, ═CHNO₂ and ═C(R^{4b})(R⁵);

R^{4a} represents, on each occasion when used above, H, C₁₋₈ alkyl or a heterocycloalkyl group, which latter two groups are optionally substituted by one or more substituents selected from G⁴, Q and B⁵ (which B⁵ group is optionally substituted by one or more substituents selected from G⁵, Q (when Q is not directly attached to an aryl or a heteroaryl group) and B⁶).

R^{4b} and R⁵ independently represent, on each occasion when used above, H or B⁴, which B⁴ group is itself optionally substituted by one or more substituents selected from G⁴, Q (when Q is not directly attached to an aryl or a heteroaryl group) and B⁵ (which B⁵ group is itself optionally substituted as described above); or when R^{4b} and/or R⁵ represent optionally substituted B⁴ groups, then any pair thereof may, for example when present on the same atom or on adjacent atoms, be linked together to form, with those, or other relevant, atoms, a 5- to 7-membered ring, optionally containing 1 to 3 heteroatoms and/or 1 to 3 double bonds, which ring is itself optionally substituted by one or more substituents selected from G⁶, Q (when the ring is not aromatic in nature) and B⁴ (which B⁴ group is optionally substituted as described above);

B⁴, B⁵ and B⁶ independently represent on each occasion when used above C₁₋₈ alkyl, heterocycloalkyl, aryl or heteroaryl;

G⁴, G⁵ and G⁶ independently represent on each occasion when used above, halo, cyano, N₃, —NO₂, —ONO₂ or -A⁷-R⁶;

wherein A⁷ represents a spacer group selected from —C(O)A⁸-, —N(R⁷)A⁹-, —OA¹⁰-, —S— or —S(O)ₙA¹¹-, in which:

A⁸ represents a single bond, —O—, —S— or —N(R⁷)—;

A⁹ represents A¹², —C(Q)S—, —S(O)ₙ—, —C(Q)O—, —S(O)ₙN(R⁷)—, —S(O)ₙO—, —C(Q)N(R⁷)C(Q)N(R⁷)—, —C(Q)N(R⁷)C(Q)O—, —C(Q)N(R⁷)S(O)ₙN(R⁷)—, —S(O)ₙN(R⁷)C(Q)N(R⁷)—, —S(O)ₙN(R⁷)C(Q)O— or —S(O)ₙN(R⁷)S(O)ₙN(R⁷)—;

A¹⁰ represents A12, —S(O)ₙ—C(Q)O—, —S(O)ₙN(R⁷)— or —S(O)ₙO—;

A¹¹ represents a single bond, —N(R⁷)— or —O—;

A¹² represents a single bond, —C(Q)- or —C(Q)N(R⁷)—;

Q represents, on each occasion when used above, a substituent connected by a double bond, which is selected from ═O, ═S, ═NR⁶, ═NN(R⁶)(R⁷), ═NOR⁶, ═NS(O)₂N(R⁶)(R⁷), ═NCN, ═CHNO₂ and ═C(R⁶)(R⁷);

R⁶ and R⁷ independently represent, on each occasion when used above, H, C₁₋₈ alkyl, heterocycloalkyl, aryl or heteroaryl, which latter four groups are optionally substituted by one or more groups selected from halo, C₁₋₆ alkyl (optionally substituted by one or more halo groups), —N(R⁸)R⁹, —OR⁸, —ONO₂ and —SR⁸; or when they do not represent H, any pair of R⁶ and R⁷ may, for example when present on the same atom or on adjacent atoms, be linked together to form, with those, or other relevant, atoms, a 5- to 7-membered ring, optionally containing 1 to 3 heteroatoms and/or 1 to 3 double bonds, which ring is itself optionally substituted by one or more groups selected from halo, C₁₋₈ alkyl (optionally substituted by one or more halo groups), —N(R⁸)R⁹, —OR⁸, —ONO₂ and —SR⁸;

R⁸ and R⁹ independently represent, on each occasion when used above, H or C₁₋₆ alkyl, which latter group is optionally substituted by one or more halo groups;

n represents, on each occasion when used above, 1 or 2; and

R^{a} and R^{b} independently represent H, halo or C₁₋₆ alkyl (which alkyl group is optionally substituted by one or more halo or C₁₋₆ alkoxy groups (which alkoxy group may itself be substituted by one or more halo group)), wherein at least one of R^{a} and R^{b} does not represent H,

or a pharmaceutically-acceptable salt thereof,

provided that, when R² and R^{a} both represent H, Y represents —C(O)—, R^{b} represents methyl and:

(i) X represents a direct bond and R³ represents methyl, then R¹ does not represent 2,6-dimethylphenyl or 2-chloro-6-methylphenyl; and

(ii) X represents —N(R^{4a})— in which R^{4a} represents H and R³ represents 4-[(2-aminosulfonyl)phenyl]phenyl, then R¹ does not represent 5-bromo-2-pyridyl,

which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl groups and C_{1-q} alkoxy groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group or a C_{2-q}-cycloalkoxy group). Further, when there is a sufficient number (i.e. a minimum of three or four as appropriate) of carbon atoms, such alkyl and alkoxy groups may also be part cyclic/acyclic. Such alkyl and alkoxy groups may also be saturated or, when there is a sufficient number (i.e. a minimum of two) of carbon atoms, be unsaturated (forming, for example in the case of the alkyl group, a C_{2-q} alkenyl or a C_{2-q} alkynyl group).

For the avoidance of doubt, alkoxy groups are attached to the rest of the molecule via the essential oxygen atom of that group.

Heterocycloalkyl groups that may be mentioned include those in which at least one (e.g. one to four) of the atoms in the ring system is other than carbon (i.e. a heteroatom, such as oxygen, nitrogen, sulphur and/or selenium), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{3-q} heterocycloalkynyl group. C_{2-q} heterocycloalkyl groups that may be mentioned include aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl, thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form.

The term “halo”, when used herein, includes fluoro, chloro, bromo and iodo.

Aryl groups that may be mentioned include C₆₋₁₃ aryl (e.g. C₆₋₁₀) groups. Such groups may be monocyclic, bicyclic or tricylic and have between 6 and 13 ring carbon atoms, in which at least one ring is aromatic. C₆₋₁₃ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are preferably linked to the rest of the molecule via an aromatic ring.

Heteroaryl groups that may be mentioned include those which have between 5 and 10 members. Such groups may be monocyclic, bicyclic or tricyclic, provided that at least one of the rings is aromatic and wherein at least one (e.g. one to four) of the atoms in the ring system is other than carbon (i.e. a heteroatom). Heterocyclic groups that may be mentioned include benzothiadiazolyl (including 2,3,1-benzothiadiazolyl), isothochromanyl and, more, preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzimidazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, more particularly, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are bicyclic or tricyclic, they are preferably linked to the rest of the molecule via an aromatic ring. Heteroaryl groups may also be in the N- or S-oxidised form.

Heteroatoms that may be mentioned include oxygen, nitrogen, sulphur and selenium.

For the avoidance of doubt, in cases in which the identity of two or more substituents in a compound of formula I may be the same, the actual identities of the respective substituents are not in any way interdependent. For example, in the situation in which R¹ and R³ are both aryl groups substituted by one or more C₁₋₈ alkyl groups, the alkyl groups in question may be the same or different. Similarly, when groups are substituted by more than one substituent as defined herein, the identities of those individual substituents are not to be regarded as being interdependent.

Compounds of the invention that may be mentioned include those in which:

R¹ does not represent pyrazolyl;

R¹ does not represent pyrimidinyl (e.g. 5-pyrimidinyl);

R¹ does not represent benzoxazolyl (e.g. 4- or 7-benzoxazolyl) or benzothiazolyl (e.g. 4- or 7-benzothiazolyl) substituted (e.g. at the 2-position) by B¹, in which B¹ represents optionally substituted aryl, heteroaryl or C₁₋₃ alkyl substituted by B², in which B² represents optionally substituted aryl or heteroaryl.

Preferred compounds of the invention include those in which:

R¹ represents aryl or heteroaryl, both of which are optionally substituted by one or two groups selected from B¹ and G¹;

R² represents H;

R³ represents C₁₋₈ alkyl, heterocycloalkyl (e.g. a five- or six-membered heterocycloalkyl group), aryl or heteroaryl, all of which are optionally substituted by one or two groups selected from B¹ and G¹;

R^{4a} represents C₁₋₆ alkyl or, preferably, H;

R^{a} and R^{b} independently represent H, C₁₋₄ alkyl or halo;

B¹ represents C₁₋₃ alkyl, aryl or heteroaryl, all of which are optionally substituted by one or more G² groups;

G¹ represents halo (e.g. fluoro, chloro or bromo), cyano or -A¹-R^{4b};

G² represents halo (e.g. fluoro);

A¹ represents —S—, —C(Z)A²-, —OA⁴- or —S(O)ₙA⁵;

A² represents —O—;

A⁴ represents A⁶ and, preferably, a single bond;

A⁵ represents a single bond;

Z represents ═S or, preferably, ═O;

R^{4b} represents B⁴

B⁴ represents C₁₋₄ alkyl or aryl, both of which groups are optionally substituted by one or more groups selected from G⁴ and B⁵;

G⁴ represents halo (e.g. chloro or fluoro);

B⁵ represents aryl (e.g. phenyl);

n represents 2.

Preferred compounds of the invention include those in which R¹ represents an optionally substituted phenyl, naphthyl, pyrrolyl, furanyl, thienyl, pyrazolyl, imidazolyl, oxazolyl, isoxazolyl, thiazolyl, pyridyl (e.g. 2-pyridyl, 3-pyridyl or 4-pyridyl), indazolyl, indolyl, indolinyl, isoindolinyl, oxindolyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, benzothiazolyl, and/or 1,4-benzodioxanyl, group. Particularly preferred values of R¹ include optionally substituted phenyl, quinolinyl (e.g. 8-quinolinyl), pyridyl, isoquinolinyl, 1,3-benzodioxolyl and 1,4-benzodioxanyl groups.

R¹ groups are preferably optionally substituted by one or more substituents selected from:

halo (e.g. fluoro or chloro);

C₁₋₃ alkyl, which alkyl group may be linear or branched (e.g. ethyl, n-propyl, isopropyl or, particularly, methyl), and/or optionally substituted by one or more halo (e.g. fluoro) group (so forming, for example, —CH₂F, —CHF₂ or, preferably, —CF₃);

More preferred optional substituents on R¹ include fluoro, chloro or trifluoromethyl groups.

Preferred compounds of the invention include those in which R³ represents an optionally substituted C₁₋₆ alkyl, phenyl, naphthyl, pyrrolidinyl, piperidinyl, piperazinyl, morpholinyl, pyrrolyl, furanyl, thienyl, pyrazolyl, imidazolyl, oxazolyl, isoxazolyl, thiazolyl, pyridyl (e.g. 2-pyridyl, 3-pyridyl or 4-pyridyl), indazolyl, indolyl, indolinyl, isoindolinyl, oxindolyl, quinolinyl, 1,2,3,4-tetrahydroquinolinyl, iso quinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, benzothiazolyl and/or benzodioxanyl group. Particularly preferred groups include an optionally substituted C₁₋₆ alkyl (such as methyl, ethyl, n-propyl, isopropyl, 71-butyl, t-butyl or hexyl), C₅₋₆ cycloalkyl (such as cyclopentyl or cyclohexyl), C₂₋₄ (e.g. C₂₋₃) alkenyl (such as propenyl), morpholinyl (such as 4-morpholinyl), piperidinyl (such as 4-piperidinyl), piperazinyl (such as 1-piperazinyl), phenyl, pyridyl (such as 2-pyridyl) or imidazolyl (such as 4-imidazolyl) group.

R³ groups are preferably optionally substituted by one or more substituents selected from:

halo (such as bromo, chloro or fluoro);

cyano;

C₁₋₆ (e.g. C₁₋₄) alkyl, which alkyl group may be linear or branched (including ethyl, propyl, butyl or, particularly, methyl) and/or optionally substituted by one or more halo (e.g. fluoro) group (so forming, for example, —CH₂F, —CHF₂ or, preferably, —CF₃);

an aryl group, such as phenyl;

a heteroaryl group, such as thienyl, pyridyl, oxazolyl or thiazolyl;

═O;

—OR¹⁰;

—C(O)OR¹¹;

—SR¹²; and/or

—S(O)₂R¹³;

wherein R¹⁰, R¹¹ and R¹² independently represent, on each occasion when used above, C₁₋₆ (e.g. C₁₋₄) allyl (such as methyl, ethyl, n-propyl, n-butyl) which alkyl group is optionally substituted by one or more halo (e.g. chloro or fluoro) atoms or aryl (e.g. phenyl) groups; and

R¹³ represents aryl (e.g. phenyl) optionally substituted by one or more halo (e.g. fluoro) atoms.

More preferred optional substituents on R³ include fluoro, chloro, bromo, 2-thienyl, phenyl, 3-chloropropylsulfanyl, ethoxycarbonyl, benzyloxycarbonyl, trifluoromethyl, methyl, methoxy, trifluoromethoxy, ethoxy, n-butoxy, cyano and 4-fluorobenzenesulfonyl groups.

More preferred compounds of the invention include those in which:

R¹ represents a phenyl group, substituted, for example in the 2- and/or 4-position by a G¹ group and/or a B¹ group. In such instances, G¹ is preferably halo (e.g. fluoro or chloro) and B¹ is preferably C₁₋₃ alkyl (e.g. methyl), which alkyl group is optionally substituted by one or more G² groups, in which G² is preferably halo (e.g. fluoro) so forming, for example a 2-chloro-4-fluorophenyl or 4-trifluoromethyl group. Alternatively, R¹ may represent a quinolinyl group, such as a 8-quinolinyl group, which group is preferably unsubstituted;

R³ represents one of the following:

(a) a C₁₋₆ alkyl group, which group is unsubstituted or substituted (e.g. at the terminal carbon atom of the alkyl group) by, for example, a G¹ or a B¹ group. In such instances B¹ is preferably an aryl (e.g. phenyl) or a heteroaryl (e.g. 2-thienyl) group and G¹ preferably represents halo (e.g. chloro or bromo) or -A¹-R^{4b}, in which A¹ represents —S— or —C(O)O—, and R^{4b} represents C₁₋₃ alkyl optionally substituted by a G⁴ group, in which G⁴ represents halo (e.g. chloro). Thus, R³ may represent ethyl, isopropyl, n-butyl, t-butyl, hexyl, 2-bromoethyl, 3-chloropropyl, 2-thien-2-ylethyl, benzyl, 2-(3-chloropropylsulfanyl)ethyl, 2-phenylethyl or acetic acid ethyl ester;
(b) a C₃ alkenyl group, which group is preferably unsubstituted, so forming, for example, a 2-propenyl (i.e. an allyl) group;
(c) a C₅₋₆ cycloalkyl group, which group is preferably unsubstituted and saturated, so forming, for example, a cyclopentyl or cyclohexyl group;
(d) a six-membered heterocycloalkyl (e.g. a piperazinyl, a piperidinyl or a morpholinyl) group, substituted in, for example, the 4-position (relative to the attachment of the R³ group to X), by a B¹ or G¹ group. In such instances, B¹ preferably represents C₁₋₃ allyl (such as methyl) and G¹ preferably represents -A¹-R^{4b}, in which A¹ represents —C(O)O— or —S(O)₂—, and R^{4b} represents C₁₋₃ alkyl (e.g. methyl), which latter group is preferably substituted by one B⁵ group, in which B⁵ preferably represents phenyl, or R^{4b} represents aryl (e.g. phenyl), which latter group is optionally substituted, for example in the 4-position of the phenyl ring, by one G⁴ group, in which G⁴ represents halo (e.g. fluoro). Thus R³ may also represent a 4-methyl-1-piperazinyl, a 4-piperidinyl-1-carboxylic acid benzyl ester, a 3-methyl-4-morpholinyl, or a 4-(4-fluorobenzenesulfonyl)-1-piperazinyl group;
(e) a phenyl group, which group is unsubstituted or is substituted in, for example, the 3- and/or the 4-position by one or two substituents selected from B¹ and G¹. In such instances B¹ may represent a C₁₋₃ alkyl (e.g. methyl) group, which group is optionally substituted by one or more G² groups, in which G² is preferably fluoro, and G¹ may represent halo, cyano or -A¹-R^{4b}, in which A¹ is preferably —O— and R^{4b} is preferably C₁₋₄ alkyl (such as methyl, ethyl or n-butyl), which alkyl group is optionally substituted by one or more fluoro groups. Thus, R³ may also represent phenyl, 4-bromophenyl, 4-chlorophenyl, 4-fluorophenyl, 4-cyanophenyl, 4-methylphenyl, 4-trifluoromethylphenyl, 3-trifluoromethylphenyl, 4-methoxyphenyl, 3,4-dimethoxyphenyl, 4-ethoxyphenyl, 4-n-butoxyphenyl or 4-trifluoromethoxyphenyl; or
(f) a pyridyl (e.g. 2-pyridyl) group, which group is preferably unsubstituted, or an imidazolyl (e.g. 4-imidazolyl) group, which group is preferably substituted, for example at the 1-position (i.e. at the secondary imidazole nitrogen), by a B¹ group, in which B¹ preferably represents a C₁₋₃ alkyl (e.g. methyl) group, so forming, for example a 1-methylimidazol-4-yl group;
   R^{a} represents H, methyl or n-butyl;
   R^{b} represents H, methyl, chloro or iodo.

Particularly preferred compounds of the invention include those of the examples described hereinafter.

Compounds of formula I may be made in accordance with techniques that are well known to those skilled in the art, for example as described hereinafter.

According to a farther aspect of the invention there is provided a process for the preparation of a compound of formula I as follows:

(i) For compounds of formula I in which R³ represents a tertiary C₁₋₈ alkyl, tertiary heterocycloalkyl, aryl or heteroaryl group and R^{b} represents C₁₋₆ alkyl, optionally substituted as hereinbefore defined, or halo, reaction of a compound corresponding to a compound of formula I in which R^{b} represents hydrogen, with an appropriate base (or a mixture of bases), such as potassium bis(trimethylsilyl)amide, sodium bis(trimethylsilyl)amide, sodium hydride, potassium tert-butoxide or an organolithium base, such as n-BuLi, s-BuLi, t-BuLi, lithium diisopropylamide or lithium 2,2,6,6-tetramethylpiperidine (which organolithium base is optionally in the presence of an additive (for example, a lithium coordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DMPU) and the like)) followed by quenching with an appropriate electrophile such as:
(a) for compounds of formula I in which R^{b} represents an optionally substituted C₁₋₆ alkyl group, an electrophile of formula II,
   R^{c}L₁ₐ II
   wherein R^{c} represents C₁₋₆ alkyl, which group is optionally substituted by one or more halo or methoxy groups and L^{1a} represents a suitable leaving group such as halo (e.g. iodo or bromo) or a sulfonate group (such as —OSO₂CF₃, OSO₂CH₃ and —OSO₂-aryl (e.g. —O-tosyl)); or
(b) for compounds of formula I in which R^{b} represents halo, an electrophile that provides a source of halide ions. For example, for bromide ions, reagents include N-bromosuccinimide, bromine and 1,2-dibromotetrachloro-ethane, for chloride ions reagents include N-chlorosuccinimide, chlorine, iodine monochloride and hexachloroethane, for iodide ions, appropriate reagents include iodine, diiodoethane and diiodotetrachloroethane and for fluoride ions reagents include xenon difluoride, SELECTFLUOR® ([1-(chloromethyl)-4-fluoro-1,4-diazonia-bicyclo[2.2.2]octane bis(tetrafluoroborate)]), CF₃OF, and perchloryl fluoride.
   This reaction may be performed in the presence of a suitable solvent, such as a polar aprotic solvent (e.g. tetrahydrofuran or diethyl ether), at sub-ambient temperatures (e.g. 0° C. to −78° C.) under an inert atmosphere.
   (ii) For compounds of formula I in which, when Y is —S(O)₂— and X is —N(R^{4a})— in which R^{4a} is B⁴, reaction of a compound of formula III, 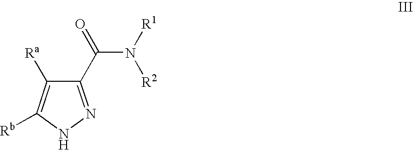
   wherein R¹, R², R^{a} and R^{b} are as hereinbefore defined, with a compound of formula IV,
   R³—X^{a}—Y-L¹ IV
   wherein, when Y represents —S(O)₂—, X^{a} represents a direct bond or —N(B⁴)—, or, for all other values of Y, X^{a} represents X as hereinbefore defined, L¹ represents a suitable leaving group, such as halo (e.g. chloro or bromo), or, when X^{a} is a direct bond, a carboxylate (e.g. a —O—C(O)—R³) group or a sulfonate (e.g. a —O—S(O)₂—R³) group, or, when X^{a} is —N(B⁴)—, an N-imidazolyl group, and R³ and Y are as hereinbefore defined, for example at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine). Preferred base/solvent systems for compounds of formula IV in which Y is —C(O)— and X is a direct bond include sodium hydride in tetrahydrofuran, DMF or mixtures thereof. Preferred base/solvent systems for compounds of formula IV in which Y is —C(O)— and X^{a} is —N(R^{4a})—, or when Y is —S(O)₂— and X^{a} is a direct bond, include dimethylaminopyridine/dichloromethane, or a mixture of triethylamine and dimethylaminopyridine in dichloromethane.
   (ii) For compounds of formula I in which X represents a single bond and Y represents —C(O)—, reaction of a compound of formula III as hereinbefore defined with a compound of formula V,
   R³C(O)OH V
   wherein R³ is as hereinbefore defined for example under similar conditions to those described under process step (ii) above, in the presence of a suitable coupling reagent (e.g. 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexafluoro-phosphate, benzotriazol-1-yloxytris-pyrrolidinophosphonium hexafluorophosphate, bromo-tris-pyrrolidinophosponium hexafluorophosphate, 2-(1H-benzotriazol-1-yl)-1,1,3,3-tetramethyluronium tetra-fluorocarbonate, 1-cyclohexylcarbodiimide-3-propyloxymethyl polystyrene, O-(7-azabenzotriazol-1-yl)-N,N,N′N′-tetramethyluronium hexafluorophosphate or O-benzotriazol-1-yl-N,N,N′,N′-tetramethyluronium tetrafluoroborate), a suitable base (e.g. as mentioned in process step (ii) above) and an appropriate solvent (e.g. as mentioned in process step (ii) above). Alternatively an azodicarboxylate may be employed under Mitsunobo conditions known to those skilled in the art.
   (iv) For compounds of formula I in which R³ represents a primary or secondary C₁₋₈ alkyl or a secondary heterocycloalk-yl group, X represents a direct bond and Y represents a —C(O)— or a —C(S)— group, reaction of a compound of formula III as hereinbefore defined with a compound of formula VI,
   R³═Y^{a} VI
   wherein Y^{a} represents either —C(O)— (i.e. C═O, so forming a ketene) or —C(S)— (i.e. C═S, so forming a thioketene) and R³ represents a primary or secondary C₁₋₈ alkyl or a secondary heterocycloalkyl group, under conditions known to those skilled in the art.
   (v) For compounds of formula I, in which X represents —NH— and Y represents —C(O)— or —C(S)—, reaction of a compound of formula III as hereinbefore defined with a compound of formula VII,
   R³N═Y^{a} VII
   wherein R³ and Yₐ are as hereinbefore defined (so forming an isocyanate or an isothiocyanate, as appropriate), under conditions known to those skilled in the art. For example, for compounds of formula VII in which Y is —C(O)—, reaction may be performed in a suitable solvent (e.g. toluene) at elevated temperature (e.g. 100° C.). For compounds of formula VII in which Y is —C(S)—, reaction may be performed in a suitable solvent (e.g. acetone) in the presence of a suitable base (e.g. potassium carbonate) at room temperature.
   (vi) For compounds of formula I in which Y represents —C(O)— or —C(S)—, reaction of a compound of formula III with:

(a) a compound of formula VIII, 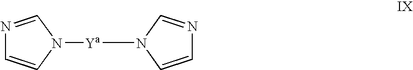
wherein, in both cases, Y^{a} is as hereinbefore defined; or
(c) when Y represents —C(O)—, triphosgene, followed by:
(1) for compounds of formula I in which X represents a direct bond, reaction with a organometallic reagent of formula X,
R³M X
wherein M represents a metal such as Mn, Fe, Ni, Cu, Zn, Pd or Ce, or a salt or complex thereof and R³ is as hereinbefore defined; or
(2) for compounds of formula I wherein X represents:
(I) —N(R^{4a})—, reaction with an amine of formula X¹,
   R³N(H)R^{4a} XI
   wherein R³ and R^{4a} are as hereinbefore defined; or
(II) a direct bond, and R³ represents a nitrogen-containing heterocycloalkyl group, in which a nitrogen atom of the heterocycloalkyl group is attached directly to the Y substituent of the compound of formula I, reaction with a corresponding secondary amine of the nitrogen-containing heterocycloalkyl group (i.e. one in which the nitrogen atom of this secondary amino group corresponds to the nitrogen atom to be attached to the Y substituent of the compound of formula I),
   in all cases under reaction condition that are known to those skilled in the art. For example, the latter reaction may be performed at below room temperature (e.g. 0° C.) in the presence of a suitable solvent (e.g. anhydrous dichloromethane).
   (vii) For compounds of formula I in which X represents —N(R^{4a})—, and R^{4a} is other than hydrogen, reaction of a corresponding compound of formula I in which X represents —N(H)— with a compound of formula XII,
   R^{4c}-L¹ XII
   wherein R^{4c} represents any value of R^{4a} mentioned hereinbefore other than H, and L¹ are as hereinbefore defined, under standard reaction conditions.
   (viii) For compounds of formula I in which Y represents —C(S)—, reaction of a corresponding compound of formula I in which Y represents —C(O)— with a suitable reagent for the conversion of a carbonyl group to a thiocarbonyl group, such as P₂S₅ or Lawesson's reagent, under conditions known to the person skilled in the art.
   (ix) Reaction of a compound of formula XIII, 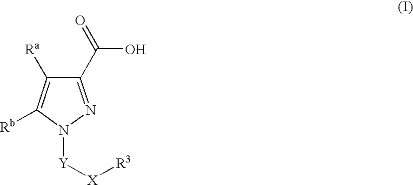
   wherein R^{a}, R^{b}, R³, Y and X are as hereinbefore defined, with a compound of formula XIV,
   HN(R¹)(R²) XIV
   wherein R¹ and R² are as hereinbefore defined under coupling conditions, for example as described in process step (iii) above. Alternatively, compounds of formula XIII may first be activated by treatment with a suitable reagent (e.g. oxalyl chloride, thionyl chloride, etc) optionally in the presence of an appropriate solvent (e.g. dichloromethane, THF, toluene or benzene) and a suitable catalyst (e.g. DMF), resulting in the formation of the respective acyl chloride. This activated intermediate may then be reacted with a compound of formula XIV under standard conditions, such as those described hereinbefore in respect of process step (ii) above. The skilled person will appreciate that when compounds of formula XIV are liquid in nature, they may serve as both solvent and reactant in this reaction. Alternative methods of performing this step include reaction of an O-protected derivative (e.g. an ethyl ester) of a compound of formula XIII with a compound of formula XIV, which latter compound may first be treated with trimethylaluminium for example in an inert atmosphere and in the presence of a suitable solvent (e.g. dichloromethane).
   (x) Reaction of a compound of formula XV,
   wherein R^{a}, R^{b}, R², R³, Y and X are as hereinbefore defined, with a compound of formula XVI,
   R¹-L² XVI
   wherein L² represents a suitable leaving group, such as halo (e.g. chloro, bromo and iodo), —OSO₂CF₃, —B(OH)₂, —Sn(R^{z})₃ (wherein R^{z} is C₁₋₆ alkyl and preferably, methyl or butyl) or —Bi(R¹)₂, and R¹ is as hereinbefore defined, for example in the presence of a catalyst containing, preferably, Pd or Cu, and a base and, optionally in the presence of solvent and a ligand. Catalysts that may be mentioned include Pd₂(dba)₃ (tris(dibenzylideneacetone)dipalladium(0)), bases that may be mentioned include cesium carbonate, ligands that may be mentioned include 2,2′-bis(diphenylphosphino)-1,1′-binaphthyl and solvents that may be employed include toluene. Such reactions may be performed at elevated temperature (e.g. at about 90° C.) under an inert (e.g. argon) atmosphere.
   (xi) For compounds of formula I in which one of R^{a} or R^{b} represents an optionally substituted C₁₋₆ alkyl group and the other represents H (as appropriate), reaction of a compound corresponding to a compound of formula I in which one of R^{a} or R^{b} represents bromo or iodo and the other represents H with a suitable organolithium base (e.g. t-BuLi, s-BuLi or n-BuLi) optionally in the presence of an additive (such as one hereinbefore described in respect of process step (i)), followed by quenching with an electrophile of formula II, as hereinbefore defined. This reaction may be performed in the presence of a suitable solvent, such as one hereinbefore described in respect of process step (i) at low temperatures (e.g. −78 to −120° C.) under an inert atmosphere.

Compounds of formula III in which R^{b} represents hydrogen and R^{a} is as hereinbefore defined may be prepared by reaction of a compound of formula XVII, 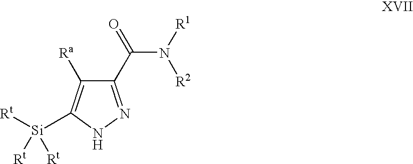
wherein each R^{t} independently represents a C₁₋₆ alkyl (e.g. a methyl or isopropyl group) or aryl (e.g. phenyl) group, and R^{a}, R¹ and R² are as hereinbefore defined, with an appropriate reagent for the removal of the silyl group, such as a source of halide anions (e.g. tetrabutylammonium fluoride, tetramethylammonium fluoride, hydrogen fluoride or potassium fluoride), for example, in the presence of a suitable solvent (e.g. tetrahydrofuran) at room temperature.

Compounds of formula III and XIII may be prepared by reaction of a compound of formula XIV as hereinbefore defined with either:
(I) a compound of formula XVIII, 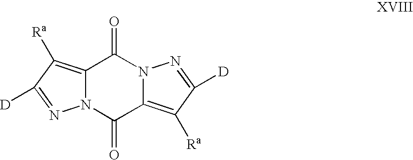
wherein D represents R^{b} or Si(R^{t})₃ (as appropriate) and R^{a}, R^{b} and R^{t} are as hereinbefore defined; or
(II) a compound of formula XIX, 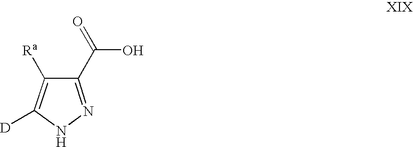
or a N-protected and/or O-protected (e.g. ester) derivative thereof, wherein R^{a} and D are as hereinbefore defined, for example under coupling conditions such as those described hereinbefore in respect of process step (ix) above.

Compounds of formula III in which R² represents H may alternatively be prepared by reaction of a compound of formula XIXA, 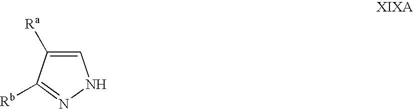
or a N-protected derivative thereof, wherein R^{a} and R^{b} are as hereinbefore defined, with a suitable base, such as one described in respect of preparation of compounds of formula I (process step (i) above), followed by reaction with a compound of formula XIXB,
R¹—N═C═O XIXB
wherein R¹ is as hereinbefore defined, followed by quenching with a suitable proton source (e.g. water or aqueous, saturated NH₄Cl solution). This reaction may be performed under similar conditions to those described above in respect of preparation of compounds of formula I (process step (i)). The skilled person will appreciate that the pyrazole nitrogen may need to be protected. The skilled person will further appreciate that the amido group will be introduced α to one of the pyrazole nitrogen atoms, and thus when R^{b} represents H, there are two alternative positions

Compounds of formula XIII may be prepared by reaction of a compound of formula XIX, in which D represents R^{b}, with an appropriate reagent under similar conditions to those described in respect any of process steps (ii) to (viii) above.

Compounds of formula XV may be prepared by reaction of a compound of formula XIII as hereinbefore defined with a compound of formula XX,
H₂NR² XX
wherein R² is as hereinbefore defined, for example under conditions such as those described hereinbefore in respect of process step (ix) above.

Compounds of formula XVIII may be prepared from compounds of formula XIX under dimerising conditions, for example in the presence of thionyl chloride (optionally in the presence of a suitable solvent and catalyst, such as one hereinbefore defined in respect of process step (ix)) at reflux. Other dimerising reagents include carbodiimides, such as 1,3-dicyclohexylcarbodiimide or 1-(3-dimethylaminopropyl)-3-ethylcarbodiimide (EDCI, or hydrochloride thereof) optionally in the presence of a suitable base (e.g. 4-dimethylaminopyridine).

Compounds of formulae III, XVIII and XIX (or derivatives thereof) in which either one of R^{a}, or R^{b} or D (as appropriate), represents halo and the other represents H, or both R^{a}, and R^{b} or D (as appropriate), represent halo, may be prepared by reaction of a compound corresponding to a compound of formula III, XVIII or XIX (as appropriate) in which R^{a}, and R^{b} or D (as appropriate), both represent H with an electrophile that provides a source of halide ions, such as one described hereinbefore in respect of process step (i)(b) above, under reaction conditions known to those skilled in the art. Thus 4-halo, 5-halo or 4,5-dihalo substituted 3-carboxylic acid pyrazoles may be prepared in such a manner.

Compounds of formula XIV in which R² represents H may be prepared:

(I) by reaction of a compound of formula XVI, as hereinbefore defined, with ammonia, or preferably with a protected derivative thereof (e.g. benzylamine), under conditions such as those described hereinbefore in respect of preparation of compounds of formula I (process step (x)); or (II) by reduction of a compound of formula XXA,
R¹—NO₂ XXA,
wherein R¹ is as hereinbefore defined, under standard reaction conditions, for example, reduction by hydrogenation in the presence of a catalyst (e.g. palladium on carbon), with a source of hydrogen (e.g. hydrogen gas or nascent hydrogen (e.g. from ammonium formate)), optionally in the presence of a solvent (such as an alcoholic solvent (e.g. methanol)).

Compounds of formula XIX (or derivatives thereof) in which D represents Si(R^{t})₃ and R^{a} represents H or R^{c} as hereinbefore defined may be prepared by reaction of a compound of formula XXI, 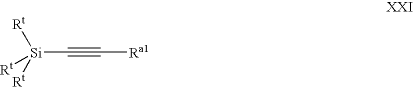
wherein R^{a1} represents H or R^{c} and R^{t} and R^{c} are as hereinbefore defined, with a compound of formula XXII,
N₂—C(H)—C(O)OH XXII
or a O-protected (e.g. ester) derivative thereof, for example at elevated temperature (e.g. at between 80 and 120° C.) for between 1 and 3 days, optionally in the presence of an inert gas and preferably without the presence of solvent.

Compounds of formula XIX (or derivatives thereof) in which D represents R^{b} and R^{b} represents R^{c} or H, may be prepared by reaction of a compound of formula XXIII, 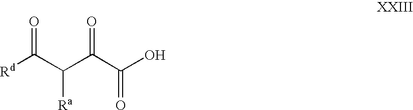
or an enol ether equivalent, or an O-protected derivative thereof, wherein R^{d} represents R^{c} or H and R^{c} and R^{a} are as hereinbefore defined, with hydrazine (or a hydrate thereof), for example in the presence of an alcoholic solvent (e.g. ethanol) at elevated temperature (e.g. at reflux).

Compounds of formula XIX in which R^{a} and D independently represent H or halo may also be prepared by reaction with a compound of formula XXIV, 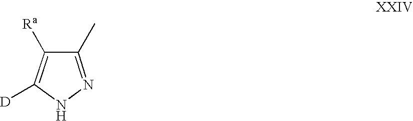
wherein R^{a} and D independently represent H or halo, under oxidising conditions known to those skilled in the art (e.g. employing an aqueous solution of potassium permanganate and heating at reflux).

Compounds of formula XIX in which one of R^{a} or D represents fluoro and the other represents H may be prepared from 4-nitropyrazole-3-carboxylic acid or 5-nitropyrazole-3-carboxylic acid (as appropriate) employing an appropriate reagent for the conversion of the nitro group to a fluoro group (such as sodium fluoride, potassium fluoride, tetramethylammonium fluoride or tetrabutylammonium fluoride) under conditions known to those skilled in the art.

Compounds of formula XIX in which one of D or R^{a} represents halo and the other represents H may be prepared from 4-nitropyrazole-3-carboxylic acid or 5-nitropyrazole-3-carboxylic acid (as appropriate) by conversion of the nitro group to an amino group (employing any suitable reducing conditions such as hydrogenation), followed by conversion of the amino group to a diazonium salt (employing reagents and conditions known to those skilled in the art, e.g. NaNO₂ and HCl at 5° C.) and then the addition of an appropriate nucleophile for the conversion to a halo group. Suitable nucleophiles for the introduction of the halo group include potassium, sodium or copper halides.

Compounds of formula XIX in which D represents halo or an optionally substituted C₁₋₆ alkyl group may be prepared from a compound corresponding to a N-protected compound of formula XIX (wherein the protective group is preferably a directing metallation group (e.g. benzenesulfonyl)) in which D represents H for example under reaction conditions and reagents such as those hereinbefore described in respect of process step (i). The skilled person will appreciate that the number of equivalents of base employed will depend upon whether the reaction is performed upon the 3-carboxylic acid or 3-carboxylic acid ester.

Compounds of formula XIX in which D represents halo may be prepared by reaction of a compound of formula XIX in which D represents —Si(R^{t})₃, or a compound corresponding to a compound of formula XIX in which the substituent D is replaced by a —Sn(R^{z})₃ (e.g. —Sn(Bu)₃) group, wherein R^{t} and R^{z} are as hereinbefore defined, with a suitable halogenating reagent such as cesium fluoride, cesium fluoroxysulfate or one described hereinbefore in respect of process step (i)(b), under reaction conditions known to those skilled in the art.

Compounds of formulae II, IV, V, VI, VII, VIII, IX, X, XI, XII, XVI, XIXA, XIXB, XX, XXA, XXI, XII, XXII and XXIV are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

The substituents R¹, R² and R³ as hereinbefore defined may be modified one or more times, after or during the processes described above for preparation of compounds of formula I by way of methods that are well known to those skilled in the art. Examples of such methods include substitutions, reductions, oxidations, alkylations, hydrolyses, esterifications, and etherifications. Further, these reactions may occur concomitantly, for example, reduction of a nitro group to an amino group may occur at the same time as reduction of a C—Br bond to a C—H bond. The precursor groups can be changed to a different such group, or to the groups defined in formula I, at any time during the reaction sequence. For example, compounds of formula I in which R³ represents a C₁₋₈ alkyl group substituted by G¹, in which G¹ represents halo may be converted to, for example, a corresponding compound of formula I in which G¹ represents -A¹-R^{4b}, such as —S—R^{4b}, by reaction with HS—R^{4b} under reaction conditions known to those skilled in the art (e.g. in the presence of a suitable base (such as triethylamine or sodium iodide) and a suitable solvent (such as dry acetone)). Further, in the case where R^{a} or R^{b} represents a halo group, such halo groups may be converted to another halo group one or more times, after or during the processes described above for the preparation of compounds of formula I. Appropriate reagents include NiCl₂ (for the conversion to a chloro group) or NiBr₂ (for the conversion to a bromo group). In this respect, the skilled person may also refer to “Comprehensive Organic Functional Group Transformations” by A. R. Katritzy, O. Meth-Cohn and C. W. Rees, Pergamon Press, 1995.

Compounds of the formula I may be isolated from their reaction mixtures using conventional techniques.

It will be appreciated by those skilled in the art that, in the processes described above and hereinafter, the functional groups of intermediate compounds may need to be protected by protecting groups. For example the pyrazole nitrogen may need to be protected. Suitable nitrogen-protecting groups include those which form:

(i) carbamate groups (i.e. alkoxy- or aryloxy-carbonyl groups);

(ii) amide groups (e.g. acetyl groups);

(iii) N-alkyl groups (e.g. hydroxymethyl or, preferably, benzyl groups);

(iv) N-sulfonyl groups (e.g. N-arylsulfonyl groups);

(v) N-phosphinyl and N-phosphoryl groups (e.g. diarylphosphinyl and diarylphosphoryl groups); or

(vi) N-silyl group (e.g. a N-trimethylsilyl group).

Further protecting groups for the pyrazole nitrogen include a methyl group, which methyl group may be deprotected under standard conditions, such as employing a pyridine hydrochloride salt at elevated temperature, for example using microwave irradiation in a sealed vessel at 200° C.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3rd edition, T. W. Greene & P. G. M. Wutz, Wiley-Interscience (1999).

The terms “primary”, “secondary” and “tertiary” when used herein assume the usual definitions known to those skilled in the art. For example, a primary group refers to a group that has two α hydrogen atoms relative to the atom of attachment of that primary group. Accordingly, a secondary group refers to one that has one a hydrogen atom and a tertiary group refers to one that has no a hydrogen atoms.

Medical and Pharmaceutical Uses

Compounds of the invention are useful because they possess pharmacological activity. Such compounds are therefore indicated as pharmaceuticals. According to a further aspect of the invention there is provided a compound of the invention for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised), may therefore be described as “prodrugs” of compounds of the invention. All prodrugs of compounds of the invention are included within the scope of the invention.

By “prodrug of a compound of the invention”, we include compounds that form a compound of the invention, in an experimentally-detectable amount, within a predetermined time (e.g. about 1 hour), following oral or parenteral administration.

Compounds of the invention are useful because, in particular, they may inhibit the activity of lipoxygenases (and particularly 15-lipoxygenase), i.e. they prevent the action of 15-lipoxygenase or a complex of which the 15-lipoxygenase enzyme forms a part and/or may elicit a 15-lipoxygenase modulating effect, for example as may be demonstrated in the test described below. Compounds of the invention may thus be useful in the treatment of those conditions in which inhibition of a lipoxygenase, and particularly 15-lipoxygenase, is required.

Compounds of the invention are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain and/or fever.

Accordingly, compounds of the invention may be useful in the treatment of asthma, chronic obstructive pulmonary disease (COPD), pulmonary fibrosis, allergic disorders, rhinitis, inflammatory bowel disease, ulcers, inflammatory pain, fever, atherosclerosis, coronary artery disease, vasculitis, pancreatitis, arthritis, osteoarthritis, rheumatoid arthritis, conjunctivitis, iritis, scleritis, uveitis, wound healing, dermatitis, eczema, psoriasis, stroke, diabetes, autoimmune diseases, Alzheimer's disease, multiple sclerosis, sarcoidosis, Hodgkin's disease and other malignancies, and any other disease with an inflammatory component.

Compounds of the invention may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds of formula I and pharmaceutically acceptable salts thereof may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a lipoxygenase (such as 15-lipoxygenase), and/or a method of treatment of a disease in which inhibition of the activity of a lipoxygenase, and particularly 15-lipoxygenase, is desired and/or required (e.g. inflammation), which method comprises administration of a therapeutically effective amount of a compound of the invention (as hereinbefore defined but without the provisos) to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or aria inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention in admixture with a pharmaceutically acceptable adjuvant, diluent or carrier.

Compounds of the invention may also be combined with other therapeutic agents that are useful in the treatment of inflammation as defined herein (e.g. NSAIDs, coxibs, corticosteroids, analgesics, inhibitors of 5-lipoxygenase, inhibitors of FLAP (5-lipoxygenase activating protein), and leukotriene receptor antagonists (LTRas), and/or other therapeutic agents that are useful in the treatment of inflammation).

According to a further aspect of the invention, there is provided a combination product comprising:
(A) a compound of the invention (as hereinbefore defined but without the provisos); and
(B) another therapeutic agent that is useful in the treatment of inflammation, wherein each of components (A) and (B) is formulated in admixture with a pharmaceutically-acceptable adjuvant, diluent or carrier.

Such combination products provide for the administration of compound of the invention in conjunction with the other therapeutic agent, and may thus be presented either as separate formulations, wherein at least one of those formulations comprises compound of the invention and at least one comprises the other therapeutic agent, or may be presented (i.e. formulated) as a combined preparation (i.e. presented as a single formulation including compound of the invention and the other therapeutic agent).

Thus, there is further provided:
(1) a pharmaceutical formulation including a compound of the invention (as hereinbefore defined but without the provisos), another therapeutic agent that is useful in the treatment of inflammation, and a pharmaceutically-acceptable adjuvant, diluent or carrier; and
(2) a kit of parts comprising components:
(a) a pharmaceutical formulation including a compound of the invention (as hereinbefore defined but without the provisos) in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

Compounds of the invention may be administered at varying doses. Oral dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For oral administration, the compositions typically contain between about 0.01 mg to about 500 mg, and preferably between about 1 mg to about 100 mg, of the active ingredient. Intravenously, the most preferred doses will range from about 0.1 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective and/or selective inhibitors of lipoxygenases, and particularly 15-lipoxygenase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the stated indications or otherwise.

Biological Test

The assay employed takes advantage of the ability of lipoxygenases to oxidize polyunsaturated fatty acids, containing a 1,4-cis-pentadiene configuration, to their corresponding hydroperoxy or hydroxyl derivatives. In this particular assay, the lipoxygenase was a purified human 15-lipoxygenase and the fatty acid was arachidonic acid. The assay is performed at room temperature (20-22° C.) and the following are added to each well in a 96-well microliter plate:

a) 35 μL phosphate buffered saline (PBS) (pH 7.4);

b) inhibitor (i.e. compound) or vehicle (0.5 μl DMSO);

c) 10 μL of a 10× concentrated solution of 15-lipoxygenase in PBS. The plates are incubated for 5 minutes at room temperature;

d) 5 μl of 0.125 mM arachidonic acid in PBS. The plate is then incubated for 10 minutes at room temperature;

e) the enzymatic reaction is terminated by the addition of 100 μl methanol; and

f) the amount of 15-hydroperoxy-eicosatetraenoic acid or 15-hydroxyeicosatetraenoic acid is measured by reverse phase HPLC.

### EXAMPLES

The invention is illustrated by way of the following examples, in which the following abbreviations may be employed:

EDCI 1-(3-dimethylaminopropyl)-3-ethyl-carbodiimide hydrochloride

DMAP 4-dimethylaminopyridine

DMF dimethylformamide

DMSO dimethylsulfoxide

EtOAc ethyl acetate

MS mass spectrum

NMR nuclear magnetic resonance

rt room temperature

TBAF tetrabutylammonium fluoride

THF tetrahydrofuran

Starting materials and chemical reagents specified in the syntheses described below are commercially available from, e.g. Sigma-Aldrich Fine Chemicals or Fisher Scientific International and its subsidiaries: Maybridge, Acros Organics etc.

Synthesis of Intermediates

### (i) 4-Methylpyrazole-3-carboxylic acid (2-chloro-4-fluorophenyl)amide

### (a) 4-Methyl-5-trimethylsilyl-pyrazole-3-carboxylic acid ethyl ester

A mixture of 1-trimethylsilyl-1-propyne (1.0 g, 8.93 mmol) and ethyl diazoacetate (1.0 g, 8.77 mmol) was stirred at 80° C. for 1 day and at 100° C. for 2 days. The mixture was diluted with EtOH:water (1:1, 10 mL) which caused precipitation of the sub-title compound as a pale yellow solid. Yield: 478 mg (24%).

¹H NMR (CDCl₃, 400 MHz) δ 10.36 (broad s, 1H), 4.30 (q, 2H), 2.34 (s, 3H), 1.25 (t, 3H), 0.30 (s, 9H).

¹³C NMR (CDCl₃, 100 MHz) δ 163.7, 143.3, 141.2, 127.7, 60.9, 14.3, 10.5, −1.5.

### (b) 4-Methyl-5-trimethylsilylpyrazole-3-carboxylic acid

NaOH (aq., 1 M, 73 mL, 73=mol) was added to a solution of 4-methyl-5-trimethylsilylpyrazole-3-carboxylic acid ethyl ester (3.3 g, 14.6 mmol) in EtOH (100 mL) at rt. The mixture was heated at 80° C. for 15 min, cooled to rt, acidified with HCl (aq, 1 M) and concentrated to near dryness. The residue was extracted with EtOAc (3×100 mL) and the combined extracts dried (NaSO₄). Concentration gave 2.5 g (86%) of the sub-title compound as a pale yellow solid.

¹H NMR (DMSO-d₆, 400 MHz) δ 11.80 (broad s, 1H), 2.27 (s, 3H), 0.30 (s, 9H).

¹³C NMR (DMSO-d₆, 100 MHz) δ 164.8, 141.8, 140.7, 126.4, 10.8, −0.5.

### (c) 3,8-Dimethyl-2,7-bis-trimethylsilyldipyrazolo[1,5-a;1′,5′-d]pyrazine-4,9-dione

A mixture of 4-methyl-5-trimethylsilylpyrazole-3-carboxylic acid (50 mg, 0.25 mmol), EDCI (73 mg, 0.38 mmol), DMAP (46 mg, 0.38 mmol) and dry CH₂Cl₂ (4 mL) was stirred at 50° C. for 2 days. The solution was diluted with CH₂Cl₂ (20 mL), washed with water (2×5 mL), filtered through silica gel and concentrated. Isohexane was added to the residue which precipitated the sub-title compound as a white solid. Yield: 31 mg (75%).

MS (M+H) 777/z 361.

¹H NMR (CDCl₃, 400 MHz) δ 2.57 (s, 6H), 0.41 (s, 18H).

¹³C NMR (CDCl₃, 100 MHz) δ 162.9, 149.5, 136.9, 130.4, 10.7, −1.3.

### (d) 4-Methyl-5-trimethylsilylpyrazole-3-carboxylic acid (2-chloro-4-fluorophenyl)amide

A mixture of 3,8-dimethyl-2,7-bis-trimethylsilyldipyrazolo[1,5-a;1′,5′-d]pyrazine-4,9-dione (31 mg, 0.093 mmol), DMAP (24 mg, 0.20 mmol) and 2-chloro-4-fluoroaniline (44 mg, 0.30 mmol) was stirred at 100° C. for 18 h. The mixture was dissolved in CH₂Cl₂ (25 mL), washed with water (2×5 mL), filtered through silica gel and concentrated. Isohexane was added to the residue which precipitated the sub-title compound as a white solid. Yield: 24 mg (80%).

MS (M⁺+H) m/z 326.

¹H NMR (DMSO-d₆, 400 MHz) δ 9.53 (s, 1H), 8.18 (dd, 1H), 7.56 (dd, 1H), 7.27 (ddd, 1H), 2.35 (s, 3H), 0.33 (s, 9H).

¹³C NMR (DMSO-d₆, 100 MHz) δ 161.7, 158.6 (d), 143.1, 142.0, 132.2, 125.5 (d), 125.4, 124.7 (d), 117.1 (d), 115.3 (d), 10.4, −0.6.

### (e) 4-Methylpyrazole-3-carboxylic acid (2-chloro-4-fluorophenyl)amide

A mixture of 4-methyl-5-trimethylsilylpyrazole-3-carboxylic acid (2-chloro-4-fluorophenyl)amide (15 mg, 0.046 mmol), TBAF (1 M in THF, 0.1 mL, 0.1 mmol) and THF (5 mL) was stirred at rt for 18 h. The mixture was concentrated, water (10 mL) was added and the mixture extracted with EtOAc (3×5 mL). The combined extracts were filtered through silica gel and concentrated. Isohexane was added to the residue which precipitated the sub-title compound as a white solid. Yield: 6 mg (51%).

MS (M⁺+H) m/z 254.

¹H NMR (CD₃CN, 400 MHz) δ 11.32 (broad s, 1H), 9.25 (s, 1H), 8.42 (dd, 1H), 7.52 (s, 1H), 7.31 (dd, 1H), 7.12 (ddd, 1H), 2.32 (d, 3H).

¹³C NMR (CD₃CN, 100 MHz) δ 161.0, 158.2 (d), 142.9, 131.9, 130.2, 124.1 (d), 122.8 (d), 118.3, 116.4 (d), 114.5 (d), 8.7.

### (ii) 4-Methyl-1H-pyrazole-3-carboxylic acid (4-trifluoromethylphenyl)-amide

### (a) 4-Methyl-5-triisopropylsilylpyrazole-3-carboxylic acid ethyl ester

Ethyl diazoacetate (1.45 g, 12.7 mmol) and 1-triisopropylsilyl-1-propyne (2.49 g, 12.7 mmol) were heated at 115° C. for 2 days in a sealed argon-filled reaction vessel. After cooling to rt, the mixture was purified by chromatography (EtOAc:heptane). Yield: 520 mg (15%) of a white solid.

¹H NMR (DMSO-d₆, 400 MHz) δ 10.0 (broad s, 1H), 4.27 (q, 2H), 2.39 (s, 3H), 1.47-1.38 (m, 6H), 1.09 (d, 18H).

### (b) 4-Methyl-5-triisopropylsilylpyrazole-3-carboxylic acid

NaOH (380 mg, 9.5 mmol) was added to a solution of 4-methyl-5-triisopropylsilylpyrazole-3-carboxylic acid ethyl ester (722 mg, 1.9 mmol) in EtOH (15 mL) and the mixture was heated at reflux for 1 h. The mixture was acidified by adding HCl (aq, 2 M). Most of the EtOH was distilled off and the aqueous residue was extracted with EtOAc (3×20 mL). The combined extracts were dried (Na₂SO₄) and concentrated to give the sub-title compound as a yellow solid in quantitative yield. The product was used in the next step without further purification.

### (c) 4-Methyl-5-triisopropylsilylpyrazole-3-carboxylic acid (4-trifluoromethylphenyl)amide

A solution of 4-methyl-5-triisopropylsilylpyrazole-3-carboxylic acid (537 mg, 1.90 mmol) in dry CH₂Cl₂ (10 mL) and dry DMF (5 mL) was added to a suspension of EDCI (546 mg, 2.85 mmol), DMAP (696 mg, 5.70 mmol) and 4-trifluoromethylaniline hydrochloride (593 mg, 3.0 mmol) in dry CH₂Cl₂ (5 mL). The mixture was stirred at reflux for 30 h under argon. The mixture was concentrated and the residue extracted with EtOAc (25 mL). The combined extracts were washed with water (2×5 mL), dried (Na₂SO₄), filtered and concentrated. The residue was purified by chromatography (EtOAc:heptane) to give the sub-title product (251 mg, 31%) as a white solid.

¹H NMR (DMSO-d₆, 400 MHz) δ 12.83 (s, 1H), 10.25 (s, 1H), 8.03 (d, 2H), 7.68 (d, 2H), 2.35 (s, 3), 1.55 (hept., 3H), 1.05 (d, 18H).

### (d) 4-Methylpyrazole-3-carboxylic acid (4-trifluoromethylphenyl)amide

TBAF (300 mg, 1.17 mmol) was added to a solution of 4-methyl-5-(triisopropylsilyl)pyrazole-3-carboxylic acid (4-trifluoromethylphenyl)amide (250 mg, 0.58 mmol) in THF (10 mL). The mixture was heated at reflux for 4 h and cooled to rt. Water (20 mL) was added and the aqueous layer was separated and extracted with EtOAc (2×20 mL). The combined extracts were dried (NaSO₄) and concentrated. The residue was purified by chromatography to give the sub-title compound, 115 mg (73%), as a solid.

¹H NMR (DMSO-d₆, 400 MHz) δ 13.20 (s, 1H), 10.30 (s, 1H), 8.25 (d, 2H), 7.70 (s, 1H), 7.66 (d, 2H), 2.26 (s, 3H).

### (iii) 4-Butylpyrazole-3-carboxylic acid (2-chloro-4-fluorophenyl)amide

### (a) 4-Butyl-5-trimethylsilylpyrazole-3-carboxylic acid ethyl ester

Ethyl diazoacetate (2.31 mL, 2.51 g, 22.0 mmol) and 1-trimethylsilyl-1-hexyne (4.04 mL, 3.09 g, 20.0 mmol) were heated at 110° C. for 2 days in a sealed argon-filled reaction vessel. The resulting red-brown liquid was purified by chromatography (heptane:EtOAc, gradient 85:15 to 80:20) to yield the sub-title compound (1.55 g, 29%) as a clear yellow syrup.

¹H NMR (DMSO-d₆, 400 MHz) δ 12.96 (s, 1H), 4.24 (q, 2H), 2.65 (t, 2H), 1.30 (m, 4H), 1.28 (t, 3H), 0.90 (t, 3H), 0.31 (s, 9H).

### (b) 4-Butylpyrazole-3-carboxylic acid

NaOH (1.4 g, 35 mmol) was added to a solution of 4-butyl-5-trimethylsilylpyrazole-3-carboxylic acid ethyl ester (1.53 g, 5.7 mmol) in absolute EtOH (15 mL) and the mixture was heated at reflux for 2 days. A yellow precipitate was formed. HCl (aq, 2 M, 18 mL) was added and the pH was adjusted to 2 with NaOH (aq, 2 M). The aqueous phase was extracted with EtOAc (100 mL) and the extract was washed with NaCl (aq, sat 40 mL). Concentration gave the sub-title product as an orange-brown solid, (1.02 g, quantitative).

¹H NMR (CDCl₃, 400 MHz) δ 13.0 (broad s, 2H), 7.52 (s, 1H), 2.65 (t, 2H), 1.50 (m, 2H), 1.30 (m, 2H), 0.88 (t, 3H).

### (c) 4-Butylpyrazole-3-carboxylic acid (2-chloro-4-fluorophenyl)amide

SOCl₂ (1.1 mL, 15=mmol) was added to an ice-cooled solution of 4-butylpyrazole-3-carboxylic acid (984 mg, 5.85 mmol) in toluene (10 mL) and CH₂Cl₂ (40 mL). The mixture was heated at reflux for 1 h and concentrated to give a brown oil. The oil was mixed with 2-chloro-4-fluoroaniline (1.8 mL, 15 mmol), DMAP (0.75 g, 6.0 mmol) and pyridine (10 mL) and heated at 100° C. for 2 days. Concentration and purification by chromatography (heptane:EtOAc, gradient 9:1 to 1:1). The solid was washed with heptane (10 mL) to give the sub-title compound as a white solid. Yield: 719 mg (42% for two steps).

¹H NMR (DMSO-d₆, 400 MHz) δ 13.24 (s, 1H), 9.52 (s, 1H), 8.17 (dd, 1H), 7.74 (s, 1H), 7.56 (dd, 1H), 7.27 (dt, 1H), 2.73 (t, 2H), 1.54 (m, 2H), 1.33 (m, 2H), 0.89 (t, 3H).

### (iv) 5-Methylpyrazole-3-carboxylic acid (2-chloro-4-fluorophenyl)amide

### (a) 5-Methylpyrazole-3-carboxylic acid ethyl ester

The title compound is commercially available (Maybridge), but has been prepared as follows (J. Med. Chem. 2002, 45, 1035). Hydrazine monohydrate (7.9 mL, 162 mmol) was added to a solution of 2,4-dioxovaleric acid ethyl ester (25.6 g, 162 mmol) in absolute EtOH (100 mL). The mixture was heated at reflux for 2 h and concentrated to give a yellow oil. Crystallisation from EtOH:water (1:3) gave the sub-title compound as colourless needles. Yield: 14.2 g (57%).

¹H NMR (DMSO-d₆, 400 MHz) δ 13.2 (broad s, 1H), 6.47 (s, 1H), 4.22 (q, 2H), 2.23 (s, 3H), 1.25 (t, 3H).

### (b) 5-Methylpyrazole-3-carboxylic acid

NaOH (6.4 g, 160 mmol) was added to a solution of 5-methylpyrazole-3-carboxylic acid ethyl ester (4.94 g, 32.0 mmol) in abs. EtOH (80 mL). The mixture was heated at reflux for 1 h and cooled to 20° C. The mixture was acidified with HCl (aq, 2 M, 85 mL, 170 mmol) and the pH adjusted to 3 with NaOH (aq, 2 M). The mixture was extracted with EtOAc (200 mL). The organic phase was washed with NaCl (aq, sat, 50 mL) and concentrated to give the title compound (3.54 g, 88%) as a white solid.

¹H NMR (DMSO-d₆, 400 MHz) δ 12.83 (broad s, 1H), 6.43 (s, 1H), 2.22 (s, 3H).

### (c) 2,7-Dimethyldipyrazolo[1,5-a:1′,5′-d]pyrazine-4,9-dione

SOCl₂ (2.2 mL, 33.8 mmol) was added to a suspension of 5-methylpyrazole-3-carboxylic acid (1.44 g, 11.4 mmol) in toluene (10 mL) and the mixture was heated at reflux for 1 h. Concentration gave the sub-title product as a yellow solid, which was used without further purification.

### (d) 5-Methylpyrazole-3-carboxylic acid (2-chloro-4-fluorophenyl)amide

A mixture of 2,7-dimethyldipyrazolo[1,5-a: 1′,5′-d]pyrazine-4,9-dione (163.8 mg, 0.76 mmol) and 2-chloro-4-fluoroaniline (1.0 mL, 8.4 mmol) was stirred at 100° C. for 3 h. The mixture was cooled to rt and a purple precipitate formed. Isohexane (10 mL), water (10 mL) and a few drops of MeOH were added and the solid was filtered off and washed with isohexane. Yield: 228 mg (60%) of a yellow solid.

¹H NMR (400 MHz, DMSO-d₆) δ 13.15 (1H, s), 9.53 (1H, s), 8.07 (1H, dd), 7.54 (1H, dd), 7.25 (1H, dt), 6.51 (1H, s), 2.28 (3H, s).

### (v) 5-Methylpyrazole-3-carboxylic acid (4-trifluoromethylphenyl) amide

The title compound was prepared as described for starting material (iv(d)) from 2,7-dimethyldipyrazolo[1,5-a:1′,5′-d]pyrazine-4,9-dione (see iv(c)) and 4-trifluoromethylaniline. Yield: 97.7 mg (20%) of light yellow solid.

¹H NMR (400 MHz, DMSO-d₆) δ 13.13 (1H, s), 10.32 (1H, s), 8.04 (2H, d), 7.66 (2H, d), 6.52 (1H, s), 2.29 (3H, s).

### Examples 1-64

### General Procedures

Method A

The relevant isocyanate (0.40 mmol) was added to a suspension of the relevant starting material (i.e. (i), (ii), (iii), (iv) or (v) above; 0.20 mmol) and K₂CO₃ (0.40 mmol) in dry acetone (20 mL) and then heated at 50° C. under argon. After the time indicated, the mixture was cooled to rt and concentrated and the residue purified by chromatography (heptane:EtOAc) to give the title compounds.

Method B

A suspension of the relevant starting material (0.20 mmol) and K₂CO₃ (0.30 mmol) in dry acetone was heated at 50° C. for 30 min and the relevant isocyanate (0.80 mmol) was added under argon. After the time indicated, the mixture was cooled to rt, diluted with EtOAc and washed with HCl (aq, 2 M) and NaCl (aq, sat). Concentration and purification by chromatography (heptane:EtOAc) gave the title compounds.

Method C

A suspension of the relevant starting material (0.20 mmol) and K₂CO₃ (0.30 mmol) in dry acetone (20 mL) was heated at reflux for 30 min and the relevant isocyanate (0.80 mmol) was added under argon. The mixture was stirred at reflux for the time indicated, cooled to rt and concentrated. The residue was suspended in CH₂Cl₂ (20 mL) and the solids were removed by filtration. Concentration and purification by chromatography (heptane:EtOAc) gave the title compounds, Concentration and purification by chromatography (heptane:EtOAc) gave the title compounds.

Method D

Triethylamine (0.20 mmol) and triphosgene (0.07 mmol) were added to a suspension of the relevant starting material (0.20 mmol) in dry CH₂Cl₂ (20 mL) under argon. The mixture was cooled to 0° C. and triethylamine (0.20 mmol) and the relevant amine (0.20 mmol) were added. The mixture was allowed to warm to rt and stirred for the indicated period of time. Concentration and purification by chromatography (heptane:EtOAc) gave the title compounds.

Method E

The relevant isocyanate (0.20 mmol) was added to a suspension of the relevant starting material (0.20 mmol) in dry toluene (20 mL) under argon. The mixture was heated at 70° C. for 18 h. Concentration and purification by chromatography (heptane:EtOAc) gave the title compounds.

Method F

The relevant starting material (0.20 mmol), the relevant sulfonyl chloride (0.40 mmol) and DMAP (0.40 mmol) were dissolved in dry acetonitrile (20 mL) under argon and the mixture was stirred in a sealed reaction vessel for the indicated time at the indicated temperature. Concentration and purification by chromatography (heptane:EtOAc) gave the title compounds.

Method G

The relevant starting material (0.20 mmol), the relevant sulfonyl chloride (0.40 mmol) and DMAP (0.40 mmol) were dissolved in dry CH₂Cl₂ (20 mL) under argon and the mixture was stirred in a sealed reaction vessel for the indicated time at the indicated temperature. Concentration and purification by chromatography (heptane:EtOAc) gave the title compounds.

Method H

The relevant starting material (0.20 mmol), the relevant sulfonyl chloride (0.40 mmol) and DMAP (0.40 mmol) were dissolved in dry 1,2-dichloroethane (20 mL) under argon and the mixture was stirred in a sealed reaction vessel for the indicated time at the indicated temperature. Concentration and purification by chromatography (heptane:EtOAc) gave the title compounds.

Method I

The relevant isothiocyanate (1.04 mmol) was added to a suspension of the relevant starting material (0.21 mmol) and K₂CO₃ (0.31 mmol) in dry acetone (2 mL) under argon. The reaction mixture was heated at 50° C. for the indicated period of time. Concentration and purification by chromatography (heptane:EtOAc) gave the title compounds.

TABLE 1
Examples (Ex.) 1-64

| | | | | | Reaction | | |
|---|---|---|---|---|---|---|---|
| | | repared from | | | conditions | | |
| | | (intermediate (i)-(v) | | | | Temp. | |
| Ex. | Chemical name | and reagent) | | Method | Time h | ° C. | Yield % |
| 1 | 4-Methylpyrazole-1,3- | i | Isopropyl- | C | 3 | 56 | 75 |
| | dicarboxylic acid 3-[(2- | | isocyanate | | | | |
| | chloro-4-fluorophenyl)- | | | | | | |
| | amide] 1-isopropylamide | | | | | | |
| 2 | 4-Methylpyrazole-1,3- | i | Cyclo- | C | 2 | 56 | 66 |
| | dicarboxylic acid 3-[(2- | | hexyliso- | | | | |
| | chloro-4-fluorophenyl)- | | cyanate | | | | |
| | amide] 1-cyclohexyl-amide | | | | | | |
| 3 | 4-Methylpyrazole-1,3- | i | 1-Hexyl- | C | 2.5 | 56 | 58 |
| | dicarboxylic acid 3-[(2- | | isocyanate | | | | |
| | chloro-4-fluorophenyl)- | | | | | | |
| | amide] 1-hexylamide | | | | | | |
| 4 | 4-Methylpyrazole-1,3- | i | 2-(2- | A | 1.5 | 50 | 77 |
| | dicarboxylic acid 3-[(2- | | Thienyl)- | | | | |
| | chloro-4-fluorophenyl)- | | ethyliso- | | | | |
| | amide] 1-[(2-thien-2- | | cyanate | | | | |
| | ylethyl)amide] | | | | | | |
| 5 | 4-Methylpyrazole-1,3- | i | 1-Allyl- | A | 0.5 | 50 | 86 |
| | dicarboxylic acid 1- | | isocyanate | | | | |
| | allylamide 3-[(2-chloro-4- | | | | | | |
| | fluorophenyl)amide] | | | | | | |
| 6 | 4-Methylpyrazole-1,3- | ii | 1-Hexyl- | C | 2 | 56 | 55 |
| | dicarboxylic acid 1- | | isocyanate | | | | |
| | hexylamide 3-[(4- | | | | | | |
| | trifluoromethylphenyl)- | | | | | | |
| | amide] | | | | | | |
| 7 | 4-Butylpyrazole-1,3- | iii | 1-Hexyl- | A | 2 | 50 | 86 |
| | dicarboxylic acid 3-[(2- | | isocyanate | | | | |
| | chloro-4-fluorophenyl)- | | | | | | |
| | amide] 1-hexylamide | | | | | | |
| 8 | 4-Butylpyrazole-1,3- | iii | Cyclo- | A | 2 | 50 | 66 |
| | dicarboxylic acid 3-[(2- | | pentyliso- | | | | |
| | chloro-4-fluorophenyl)- | | cyanate | | | | |
| | amide] 1-cyclopentylamide | | | | | | |
| 9 | 4-Butylpyrazole-1,3- | iii | Isopropyl- | A | 2 | 50 | 66 |
| | dicarboxylic acid 3-[(2- | | isocyanate | | | | |
| | chloro-4-fluorophenyl)- | | | | | | |
| | amide] 1-isopropylamide | | | | | | |
| 10 | 4-Methylpyrazole-1,3- | ii | Cyclo- | C | 2 | 56 | 23 |
| | dicarboxylic acid 1- | | hexyliso- | | | | |
| | cyclohexylamide 3-[(4- | | cyanate | | | | |
| | trifluoromethylphenyl)- | | | | | | |
| | amide] | | | | | | |
| 11 | 4-Butylpyrazole-1,3- | iii | Cyclo- | C | 1 | 50 | 26 |
| | dicarboxylic acid 3-[(2- | | hexyliso- | | | | |
| | chloro-4-fluorophenyl)- | | cyanate | | | | |
| | amide] 1-cyclohexylamide | | | | | | |
| 12 | 4-Methylpyrazole-1,3- | i | 2-Bromo- | A | 1 | 50 | 53 |
| | dicarboxylic acid 1-[(2- | | ethyliso- | | | | |
| | bromoethyl)-amide] 3-[(2- | | cyanate | | | | |
| | chloro-4-fluorophenyl)- | | | | | | |
| | amide] | | | | | | |
| 13 | 4-Methylpyrazole-1,3- | i | 3-Chloro- | A | 1 | 50 | 65 |
| | dicarboxylic acid 3-[(2- | | propyliso- | | | | |
| | chloro-4-fluorophenyl)- | | cyanate | | | | |
| | amide] 1-[(3-chloro- | | | | | | |
| | propyl)amide] | | | | | | |
| 14 | 4-Methylpyrazol-1,3- | i | Benzyliso- | A | 1 | 50 | 75 |
| | dicarboxylic acid 1- | | cyanate | | | | |
| | benzylamide 3-[(2-chloro- | | | | | | |
| | 4-fluorophenyl)-amide] | | | | | | |
| 15 | 4-Methylpyrazole-1,3- | i | Ethyliso- | C | 2 | 56 | 25 |
| | dicarboxylic acid 3-(2- | | cyanate | | | | |
| | chloro-4- | | | | | | |
| | fluorophenylamide) 1- | | | | | | |
| | ethylamide | | | | | | |
| 16 | 4-Methylpyrazole-1,3- | i | Phenethyl- | A | 1 | 50 | 98 |
| | dicarboxylic acid 3-[(2- | | isocyanate | | | | |
| | chloro-4-fluorophenyl)- | | | | | | |
| | amide] 1-(phenethyl- | | | | | | |
| | amide) | | | | | | |
| 17 | {[3-(2-Chloro-4- | i | Isocyanatoacetic | A | 0.25 | 50 | 75 |
| | fluorophenylcarbamoyl)-4- | | acid | | | | |
| | methylpyrazole-1- | | ethyl ester | | | | |
| | carbonyl]-amino}acetic | | | | | | |
| | acid ethyl ester | | | | | | |
| 18 | 4-{[3-(2-Chloro-4- | i | 4-Isocyanatopiperidine- | A | 0.25 | 50 | 80 |
| | fluorophenylcarbamoyl)-4- | | 1- | | | | |
| | methylpyrazole-1- | | carboxylic | | | | |
| | carbonyl]-amino}- | | acid benzyl | | | | |
| | piperidine-1-carboxylic | | ester | | | | |
| | acid benzyl ester | | | | | | |
| 19 | 4-Methyl-1-(4-methyl- | i | 1-Methyl- | D | 1 | 20 | 75 |
| | piperazine-1-carbonyl)- | | piperazine | | | | |
| | pyrazole-3-carboxylic acid | | | | | | |
| | (2-chloro-4-fluorophenyl)- | | | | | | |
| | amide | | | | | | |
| 20 | 5-Methylpyrazole-1,3- | iv | tert- | E | 18 | 70 | 3 |
| | dicarboxylic acid 1-tert- | | Butyliso- | | | | |
| | butylamide 3-[(2-chloro-4- | | cyanate | | | | |
| | fluorophenyl)amide] | | | | | | |
| 21 | 5-Methylpyrazole-1,3- | iv | Isopropyl- | E | 18 | 70 | 5 |
| | dicarboxylic acid 3-[(2- | | isocyanate | | | | |
| | chloro-4-fluorophenyl)- | | | | | | |
| | amide] 1-isopropylamide | | | | | | |
| 22 | 5-Methylpyrazole-1,3- | iv | Cyclo- | A | 2 | 50 | 44 |
| | dicarboxylic acid 3-[(2- | | hexyliso- | | | | |
| | chloro-4-fluorophenyl)- | | cyanate | | | | |
| | amide] 1-cyclohexylamide | | | | | | |
| 23 | 5-Methylpyrazole-1,3- | v | Cyclo- | A | 2 | 50 | 39 |
| | dicarboxylic acid 1- | | hexyliso- | | | | |
| | cyclohexylamide 3-[(4- | | cyanate | | | | |
| | trifluoromethylphenyl)- | | | | | | |
| | amide] | | | | | | |
| 24 | 5-Methylpyrazole-1,3- | v | 1-Hexyl- | A | 1 | 50 | 40 |
| | dicarboxylic acid 1- | | isocyanate | | | | |
| | hexylamide 3-[(4- | | | | | | |
| | trifluoromethylphenyl)- | | | | | | |
| | amide] | | | | | | |
| 25 | 5-Methylpyrazole-1,3- | v | Isopropyl- | A | 2 | 50 | 61 |
| | dicarboxylic acid 1- | | isocyanate | | | | |
| | isopropylamide 3-[(4- | | | | | | |
| | trifluoromethylphenyl)- | | | | | | |
| | amide] | | | | | | |
| 26 | 5-Methylpyrazole-1,3- | iv | 1-Hexyl- | A | 1.5 | 50 | 67 |
| | dicarboxylic acid 3-[(2- | | isocyanate | | | | |
| | chloro-4-fluorophenyl)- | | | | | | |
| | amide] 1-hexylamide | | | | | | |
| 27 | 5-Methylpyrazole-1,3- | v | 2-Bromo- | A | 0.5 | 50 | 39 |
| | dicarboxylic acid 1-[(2- | | ethyliso- | | | | |
| | bromoethyl)-amide] 3-[(4- | | cyanate | | | | |
| | trifluoromethyl-phenyl)- | | | | | | |
| | amide] | | | | | | |
| 28 | 5-Methylpyrazole-1,3- | iv | Ethyliso- | A | 1 | 50 | 66 |
| | dicarboxylic acid 3-[(2- | | cyanate | | | | |
| | chloro-4-fluorophenyl)- | | | | | | |
| | amide] 1-ethylamide | | | | | | |
| 29 | 5-Methylpyrazole-1,3- | iv | Phenethyl- | A | 2.5 | 50 | 41 |
| | dicarboxylic acid 3-[(2- | | isocyanate | | | | |
| | chloro-4-fluorophenyl)- | | | | | | |
| | amide] 1-(phenethyl- | | | | | | |
| | amide) | | | | | | |
| 30 | 5-Methylpyrazole-1,3- | iv | 2-(2- | A | 0.5 | 50 | 44 |
| | dicarboxylic acid 3-[(2- | | Thienyl)- | | | | |
| | chloro-4-fluorophenyl)- | | ethyliso- | | | | |
| | amide] 1-[(2-thien-2- | | cyanate | | | | |
| | ylethyl)-amide] | | | | | | |
| 31 | {[3-(2-Chloro-4- | iv | Isocyanatoacetic | A | 0.5 | 50 | 40 |
| | fluorophenylcarbamoyl)-5- | | acid | | | | |
| | Methylpyrazole-1- | | ethyl ester | | | | |
| | carbonyl]-amino}-acetic | | | | | | |
| | acid ethyl ester | | | | | | |
| 32 | 4-{[3-(2-Chloro-4- | iv | 4-Isocyanatopiperidine- | A | 2 | 50 | 57 |
| | fluorophenylcarbamoyl)-5- | | 1- | | | | |
| | methylpyrazole-1- | | carboxylic | | | | |
| | carbonyl]amino}- | | acid benzyl | | | | |
| | piperidine-1-carboxylic | | ester | | | | |
| | acid benzyl ester | | | | | | |
| 33 | 5-Methylpyrazole-1,3- | iv | 1-Allyl- | B | 1.5 | 50 | 6 |
| | dicarboxylic acid 1- | | isocyanate | | | | |
| | allylamide 3-[(2-chloro-4- | | | | | | |
| | fluorophenyl)amide] | | | | | | |
| 34 | 5-Methyl-1-(4-methyl- | iv | 1-Methyl- | D | 1 | 20 | 17 |
| | piperazine-1-carbonyl)- | | piperazine | | | | |
| | pyrazole-3-carboxylic acid | | | | | | |
| | (2-chloro-4- | | | | | | |
| | fluorophenyl)amide | | | | | | |
| 35 | 1-Benzenesulfonyl-4- | i | Benzene- | G | 18 | 100 | 28 |
| | methylpyrazole-3- | | sulfonyl | | | | |
| | carboxylic acid (2-chloro- | | chloride | | | | |
| | 4-fluorophenyl)amide | | | | | | |
| 36 | 4-Methyl-1-(3- | i | 3-Trifluoro- | G | 18 | 100 | 36 |
| | trifluoromethyl- | | methyl- | | | | |
| | benzenesulfonyl)-pyrazole- | | benzene- | | | | |
| | 3-carboxylic acid (2- | | sulfonyl | | | | |
| | chloro-4-fluorophenyl)- | | chloride | | | | |
| | amide | | | | | | |
| 37 | 1-(4-Bromo- | i | 4-Bromo- | G | 12 | 100 | 16 |
| | benzenesulfonyl)-4- | | benzene- | | | | |
| | methylpyrazole-3- | | sulfonyl | | | | |
| | carboxylic acid (2-chloro- | | chloride | | | | |
| | 4-fluorophenyl)-amide | | | | | | |
| 38 | 4-Methyl-1-(toluene-4- | i | Toulene-4- | G | 18 | 100 | 37 |
| | sulfonyl)pyrazole-3- | | sulfonyl | | | | |
| | carboxylic acid (2-chloro- | | chloride | | | | |
| | 4-fluorophenyl)-amide | | | | | | |
| 39 | 1-(4-Methoxy- | i | 4-Methoxy- | G | 18 | 100 | 44 |
| | benzenesulfonyl)-4- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 40 | 1-(4-Butoxy- | i | 4-Butoxy- | G | 18 | 100 | 9 |
| | benzenesulfonyl)-4- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 41 | 1-(3,4-Dimethoxy- | i | 3,4-Dimethoxy- | H | 1.5 | 100 | 45 |
| | benzenesulfonyl)-4- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 42 | 1-(4-Chloro- | i | 4-Chloro- | H | 1.5 | 120 | 56 |
| | benzenesulfonyl)-4- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 43 | 1-(4-Ethoxy- | i | 4-Ethoxy- | F | 2.5 | 80 | 78 |
| | benzenesulfonyl)-4- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 44 | 4-Methyl-1-(4- | i | 4-Trifluoro- | F | 3 | 80 | 89 |
| | trifluoromethoxy- | | methoxy- | | | | |
| | benzenesulfonyl)-pyrazole- | | benzene- | | | | |
| | 3-carboxylic acid (2- | | sulfonyl | | | | |
| | chloro-4-fluorophenyl)- | | chloride | | | | |
| | amide | | | | | | |
| 45 | 4-Methyl-1-(4- | i | 4-Trifluoro- | F | 4 | 50 | 86 |
| | trifluoromethyl- | | methyl- | | | | |
| | benzenesulfonyl)-pyrazole- | | benzene- | | | | |
| | 3-carboxylic acid (2- | | sulfonyl | | | | |
| | chloro-4-fluorophenyl)- | | chloride | | | | |
| | amide | | | | | | |
| 46 | 1-(4-Fluoro- | i | 4-Fluoro- | F | 1 | 70 | 40 |
| | benzenesulfonyl)-4- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 47 | 4-Methyl-1-(1-methyl- | i | 1-Methyl- | F | 2 | 80 | 51 |
| | imidazole-4-sulfonyl)- | | imidazole- | | | | |
| | pyrazole-3-carboxylic acid | | 4-sulfonyl | | | | |
| | (2-chloro-4-fluorophenyl)- | | chloride | | | | |
| | amide | | | | | | |
| 48 | 1-(4-Cyano- | i | 4-Cyano- | F | 2 | 50 | 23 |
| | benzenesulfonyl)-4- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 49 | 1-(4-Bromo- | iv | 4-Bromo- | G | 12 | 100 | 55 |
| | benzenesulfonyl)-5- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 50 | 1-(4-Butoxy- | iv | 4-Butoxy- | F | 3 | 100 | 52 |
| | benzenesulfonyl)-5- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 51 | 1-(4-Methoxy- | iv | 4-Methoxy- | H | 3 | 100 | 38 |
| | benzenesulfonyl)-5- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 52 | 5-Methyl-1-(4- | iv | 4-Trifluoro- | H | 3 | 120 | 16 |
| | trifluoromethoxy- | | methoxy- | | | | |
| | benzenesulfonyl)-pyrazole- | | benzene- | | | | |
| | 3-carboxylic acid (2- | | sulfonyl | | | | |
| | chloro-4-fluorophenyl)- | | chloride | | | | |
| | amide | | | | | | |
| 53 | 5-Methyl-1-(toluene-4- | iv | Toluene-4- | F | 1.5 | 90 | 81 |
| | sulfonyl)pyrazole-3- | | sulfonyl | | | | |
| | carboxylic acid (2-chloro- | | chloride | | | | |
| | 4-fluorophenyl)amide | | | | | | |
| 54 | 5-Methyl-1-(3- | iv | 3-Trifluoro- | F | 1.5 | 90 | 60 |
| | trifluoromethyl- | | methyl- | | | | |
| | benzenesulfonyl)pyrazole- | | benzene- | | | | |
| | 3-carboxylic acid | | sulfonyl | | | | |
| | (2-chloro-4-fluorophenyl)- | | chloride | | | | |
| | amide | | | | | | |
| 55 | 1-Benzenesulfonyl-5- | iv | Benzene- | F | 18 | 55 | 30 |
| | methylpyrazole-3- | | sulfonyl | | | | |
| | carboxylic acid (2-chloro- | | chloride | | | | |
| | 4-fluorophenyl)amide | | | | | | |
| 56 | 1-(4-Chloro- | iv | 4-Chloro- | F | 1.5 | 90 | 70 |
| | benzenesulfonyl)-5- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 57 | 1-(4-Ethoxy- | iv | 4-Ethoxy- | F | 1.5 | 80 | 42 |
| | benzenesulfonyl)-5- | | benzene- | | | | |
| | methylpyrazole-3- | | sulfonyl | | | | |
| | carboxylic acid (2-chloro- | | chloride | | | | |
| | 4-fluorophenyl)amide | | | | | | |
| 58 | 1-(3,4-Dimethoxy- | iv | 3,4- | F | 2 | 80 | 8 |
| | benzenesulfonyl)-5- | | Dimethoxy- | | | | |
| | methylpyrazole-3- | | benzene- | | | | |
| | carboxylic acid (2-chloro- | | sulfonyl | | | | |
| | 4-fluorophenyl)amide | | chloride | | | | |
| 59 | 5-Methyl-1-(4-trifluoro- | iv | 4-Trifluoro- | F | 20 | 55 | 25 |
| | methyl-benzenesulfonyl)- | | methyl- | | | | |
| | pyrazole-3-caroboxylic acid | | benzene- | | | | |
| | (2-chloro-4-fluoro- | | sulfonyl | | | | |
| | phenyl)amide | | chloride | | | | |
| 60 | 5-Methyl-1-(1-methyl- | iv | 1-Methyl- | F | 18 | 80 | 51 |
| | imidazole-4-sulfonyl)- | | imidazole- | | | | |
| | pyrazole-3-carboxylic acid | | 4-sulfonyl | | | | |
| | (2-chloro-4-fluorophenyl)- | | chloride | | | | |
| | amide | | | | | | |
| 61 | 1-Ethylthiocarbamoyl-4- | i | Ethyl | I | 2 | 50 | 41 |
| | methylpyrazole-3- | | isothio- | | | | |
| | carboxylic acid (2-chloro- | | cyanate | | | | |
| | 4-fluorophenyl)amide | | | | | | |
| 62 | 1-Butylthiocarbamoyl-4- | i | 1-Butyl | I | 18 | 50 | 61 |
| | methylpyrazole-3- | | isothio- | | | | |
| | carboxylic acid (2-chloro- | | cyanate | | | | |
| | 4-fluorophenyl)amide | | | | | | |
| 63 | 4-Methyl-1- | i | Phenethyl | I | 1.5 | 50 | 16 |
| | phenethylthiocarbamoyl- | | isothio- | | | | |
| | pyrazole-3-carboxylic acid | | cyanate | | | | |
| | (2-chloro-4- | | | | | | |
| | fluorophenyl)amide | | | | | | |
| 64 | 1-Ethylthiocarbamoyl-5- | iv | Ethyl | I | 18 | 50 | 6 |
| | methylpyrazole-3- | | isothio- | | | | |
| | carboxylic acid (2-chloro- | | cyanate | | | | |
| | 4-fluorophenyl)amide | | | | | | |

### Example 65

### 4-Methyl-1-(pyridine-2-sulfonyl)pyrazole-3-carboxylic acid (2-chloro-4-fluorophenyl)amide

### (a) Pyridine-2-sulfonyl bromide

Bromine (˜2 mL, ˜40 mmol) was added dropwise to a solution of 2-mercaptopyridine (1.0 g, 9.0 mmol) in acetic acid:water (7:3, 20 mL) at −5 to −10° C. until the orange colour persisted. A precipitate was formed and the mixture was stirred at −5 to −10° C. for 30 minutes and concentrated under high vacuum (oil pump, temperature below 30° C.) to give an orange oil. The oil was triturated with diethyl ether to give a semisolid which solidified in the freezer at −18° C. The NMR indicated that the material was a 1:1 mixture of the sulfonic acid and the sulfonyl bromide. The mixture was used in the next step without further purification.

### (b) 4-Methyl-1-(pyridine-2-sulfonyl)pyrazole-3-carboxylic acid (2-chloro-4-fluorophenyl)amide

A mixture of the starting material (i) (120 mg, 0.5 mmol) and the impure sulfonyl bromide (1.0 g, 2.2 mmol; see step (a)) in acetonitrile (5 mL) was heated at reflux for 18 h. The mixture was concentrated and the residue washed with cold acetonitrile to give the title product as a hydrobromide salt. Yield: 75 mg (38%) of a brown solid.

### Example 66

### 1-Benzenesulfonyl-5-chloropyrazole-3-carboxylic acid (2-chloro-4-fluorophenyl)amide

### (a) Dipyrazolo[1,5-a;1′,5′-d]pyrazine-4,9-dione

DMF (0.1 mL, 1.4 mmol) was added dropwise to a stirred suspension of pyrazole-3-carboxylic acid (5.0 g, 44.6 mmol) in SOCl₂ (40 mL). The mixture was heated at reflux for 48 h and concentrated to give a white solid which was used without further purification.

### (b) Pyrazole-3-carboxylic acid (2-chloro-4-fluorophenyl)amide

The sub-title compound was prepared as described for starting material (iv(d)) from dipyrazolo[1,5-a:1′,5′-d]pyrazine-4,9-dione (see (a) above) and 2-chloro-4-fluoroaniline. Yield: 222 mg (61%) as a white solid.

MS (M⁺+H) 71/z 240.

¹H NMR (CD₃CN, 400 MHz) δ 11.56 (broad s, 1H), 9.21 (s, 1H), 8.39 (dd, 1H), 7.74 (d, 1H), 7.34 (dd, 1H), 7.14 (ddd, 1H), 6.83 (d, 1H).

### (c) 1-Benzenesulfonylpyrazole-3-carboxylic acid (2-chloro-4-fluorophenyl)amide

The sub-title compound was prepared according to the general procedure (F) from pyrazole-3-carboxylic acid (2-chloro-4-fluorophenyl)amide (1.50 g, 6.27 mmol; see (b) above) and benzenesulfonyl chloride (1.6 mL, 12.54 mmol). The mixture was stirred at 80° C. for 5 h. Yield: 1.79 g (75%) of a white solid.

MS (M⁺+H) m/z 419.

¹H NMR (DMSO-d₆, 400 MHz) δ 10.0 (s, 1H), 8.67 (d, 1H), 8.11 (d, 1H), 7.85 (tt, 1H), 7.72 (t, 1H), 7.69 (dd, 1H), 7.55 (dd, 1H), 7.26 (dt, 1H), 7.06 (d, 1H).

### (d) 1-Benzenesulfonyl-5-chloropyrazole-3-carboxylic acid (2-chloro-4-fluorophenyl)amide

n-BuLi (1.5 M in hexane, 0.73 mL, 1.10 mmol) was added dropwise under argon to a stirred solution of 1-benzenesulfonylpyrazole-3-carboxylic acid (2-chloro-4-fluorophenyl)amide (190 mg, 0.50 mmol) in dry THF (9 mL) at −78° C. After 20 min a suspension of N-chlorosuccinimide (93 mg, 0.70 mmol) in THF (1 mL) was added dropwise. After 2 h at −78° C., the mixture was poured into a mixture of HCl (aq, 0.1 M, 5 mL) and NaCl (aq, sat, 25 μL). The aqueous phase was extracted with EtOAc (3×10 mL), the combined extracts were concentrated and the residue purified by chromatography (heptane:CH₂Cl₂:triethylamine, 50:50:1). Yield: 29 mg (14%) of a white solid.

### Example 67

### 1-Benzenesulfonyl-5-iodopyrazole-3-carboxylic acid (2-chloro-4-fluoro-phenyl)-amide

n-BuLi (1.3 M in hexane, 485 μL, 0.63 mmol) was added dropwise under argon to a stirred solution of 1-benzenesulfonylpyrazole-3-carboxylic acid (2-chloro-4-fluorophenyl)amide (see example 66(c), 114 mg, 0.30 mmol) in dry THF (10 mL) at −78° C. The yellow solution was stirred for 30 min at −78° C. Iodine (189 mg, 0.74 mmol) in THF (0.5 mL) was added and the mixture was stirred for 2 h at −78° C. The reaction was quenched with NH₄Cl (aq, sat) and extracted with EtOAc (4×20 mL). The combined extracts were dried (Na₂SO₄) and concentrated. Purification by chromatography (heptane:EtOAc) gave 126 mg (83%) of the title compound as a white solid.

### Example 68

### 4-Methylpyrazole-1,3-dicarboxylic acid 3-[(2-chloro-4-fluorophenyl)amide]1-{[2-(3-chloropropylsulfanyl) ethyl]amide}

Triethylamine (16 μl, 12 mg, 0.12 mmol) and 3-chloropropan-1-thiol (15 μl, 17 mg, 0.15 mmol) were added to a solution of 4-methylpyrazole-1,3-dicarboxylic acid 1-[(2-bromoethyl)amide]3-[(2-chloro-4-fluorophenyl)amide] (Example 12, 42 mg, 0.10 mmol) and sodium iodide (17 mg, 0.11 mmol) in dry acetone (2 mL). The mixture was stirred at rt overnight and concentrated. Purification by chromatography (heptane:EtOAc, gradient 85:15 to 0:100) gave the title compound as a white solid: 3 mg (7%).

TABLE 2
Physical properties of the compounds of Examples 1 to 68

| | | MS | |
|---|---|---|---|
| | | (M⁺ + 1), | |
| Ex. | M.W. | m/z | ¹H NMR (DMSO-d₆, 400 MHz), δ |
| 1 | 338.77 | 339 | 9.86 (1H, s), 8.21 (1H, s), 8.19 (1H, d), 7.72 (1H, dd), |
| | | | 7.60 (1H, dd), 7.30 (1H, dt), 4.04-3.95 (1H, m), 2.25 (3H, s), |
| | | | 1.25 (6H, d) |
| 2 | 378.83 | 379 | 9.88 (1H, s), 8.21 (1H, s), 8.16 (1H, d), 7.72 (1H, dd), |
| | | | 7.60 (1H, dd), 7.30 (1H, dt), 3.70-3.61 (1H, m), 2.24 (3H, s), |
| | | | 1.95-1.14 (10H, m) |
| 3 | 380.85 | 381 | 9.74 (1H, s), 8.57 (1H, t), 8.21 (1H, s), 7.81 (1H, dd), |
| | | | 7.60 (1H, dd), 7.30 (1H, dt), 3.29-3.27 (1H, m), 2.25 (3H, s), |
| | | | 1.56 (2H, quint.), 1.4-1.2 (8H, m), 0.88-0.85 (3H, m) |
| 4 | 406.86 | 407 | 9.69 (1H, s), 8.67 (1H, t), 8.23 (1H, s), 7.85 (1H, dd), |
| | | | 7.60 (1H, dd), 7.34 (1H, dd), 7.30 (1H, dt), 6.97-6.93 (2H, m), |
| | | | 3.55 (2H, q), 3.12 (2H, t), 2.26 (3H, s) |
| 5 | 336.75 | 337 | 9.77 (1H, s), 8.72 (1H, t), 8.23 (1H, s), 7.79 (1H, dd), |
| | | | 7.60 (1H, dd), 7.30 (1H, dt), 5.98-5.87 (1H, m), 5.17 (2H, 2 dd), |
| | | | 3.95 (2H, t), 2.26 (3H, s) |
| 6 | 396.41 | 397 | 10.32 (1H, s), 8.54 (1H, t), 8.22 (1H, s), 7.97 (2H, d), |
| | | | 7.76 (2H, d), 3.34-3.30 (2H, m), 2.26 (3H, s), 1.62-1.55 (2H, m), |
| | | | 1.37-1.23 (6H, m), 0.89-0.85 (3H, m) |
| 7 | 422.93 | 423 | 9.76 (1H, s), 8.52 (1H, t), 8.20 (1H, s), 7.80 (1H, dd), |
| | | | 7.60 (1H, dd), 7.30 (1H, dt), 3.29 (2H, q), 2.71 (2H, t), |
| | | | 1.60-1.50 (4H, m), 1.35-1.24 (8H, m), 0.91-0.84 (6H, m) |
| 8 | 406.89 | 407 | 9.89 (1H, s), 8.20 (1H, d), 8.19 (1H, s), 7.72 (1H, dd), |
| | | | 7.61 (1H, dd), 7.31 (1H, dt), 4.12 (1H, sext.), 2.71 (2H, t), |
| | | | 2.01-1.93 (2H, m), 1.76-1.68 (2H, m), 1.64-1.54 (4H, m), |
| | | | 1.54 (2H, quint.), 1.31 (2H, sext.), 0.88 (3H, t) |
| 9 | 380.85 | 381 | 9.88 (1H, s), 8.20 (1H, d) 8.19 (1H, s), 7.72 (1H, dd), |
| | | | 7.61 (1H, dd), 7.31 (1H, dt), 3.99 (1H, oct.), 2.70 (2H, t), 1.54 (2H, |
| | | | quint.), 1.31 (2H, sext.), 1.26 (6H, d), 0.88 (3H, t) |
| 10 | 394.40 | 395 | 10.36 (s, 1H), 8.23 (s, 1H), 8.16 (d, s 1H), 7.99 (d, 2H), |
| | | | 7.75 (d, 2H), 3.71-3.62 (m, 1H), 2.27 (s, 3H), 1.95-1.00 (m, 10H) |
| 11 | 420.91 | 421 | 9.89 (1H, s), 8.19 (1H, s), 8.18 (1H, d), 7.70 (1H, dd), |
| | | | 7.62 (1H, dd), 7.31 (1H, dt), 3.68-3.61 (1H, m), 2.70 (2H, t), |
| | | | 1.90-1.10 (10H, m), 1.54 (2H, quint.), 1.31 (2H, sext.), 0.88 (3H, t) |
| 12 | 403.64 | 405 | 9.72 (1H, s), 8.76 (1H, t), 8.26 (1H, s), 7.83 (1H, dd), |
| | | | 7.61 (1H, dd), 7.31 (1H, dt), 3.71 (2H, q), 3.66 (2H, t), 2.26 (3H, s) |
| 13 | 373.21 | 373 | 9.71 (1H, s), 8.61 (1H, t), 8.23 (1H, s), 7.81 (1H, dd), |
| | | | 7.61 (1H, dd), 7.30 (1H, dt), 3.72 (2H, t), 3.43 (2H, q), 2.26 (3H, |
| | | | s), 2.04 (2H, quint.) |
| 14 | 386.81 | 387 | 9.75 (1H, s), 9.07 (1H, t), 8.26 (1H, s), 7.79 (1H, dd), |
| | | | 7.59 (1H, dd), 7.38-7.27 (6H, m), 4.54 (2H, d), 2.27 (3H, s) |
| 15 | 324.74 | 325 | 9.73 (1H, s), 8.53 (1H, t), 8.21 (1H, s), 7.80 (1H, dd), |
| | | | 7.59 (1H, dd), 7.30 (1H, dt), 3.40-3.37 (2H, m), 2.25 (3H, s), |
| | | | 1.18 (3H, t) |
| 16 | 400.84 | 401 | 9.71 (1H, s), 8.61 (1H, t), 8.22 (1H, s), 7.84 (1H, dd), |
| | | | 7.61 (1H, dd), 7.33-7.20 (6H, m), 3.53 (2H, q), 2.90 (2H, t), |
| | | | 2.26 (3H, s) |
| 17 | 382.78 | 383 | 9.73 (1H, s), 8.92 (1H, t), 8.27 (1H, s), 7.86 (1H, dd), |
| | | | 7.61 (1H, dd), 7.30 (1H, dt), 4.14 (2H, q), 4.11 (2H, d), 2.27 (3H, |
| | | | s), 1.22 (3H, t) |
| 18 | 513.96 | 514 | 9.80 (1H, s), 8.33 (1H, d), 8.23 (1H, s), 7.73 (1H, dd), |
| | | | 7.62 (1H, dd), 7.36 (5H, broad s), 7.31 (1H, dt), 5.08 (2H, s), |
| | | | 4.01-1.23 (9H, m), 2.24 (3H, s) |
| 19 | 379.82 | 380 | 9.59 (1H, s), 8.04 (1H, dd), 7.58 (1H, dd), 7.29 (1H, dt), |
| | | | 3.71 (4H, broad s), 2.43 (4H, t), 2.27 (3H, s), 2.22 (3H, s) |
| 20 | 352.80 | 353 | 13.16 (1H, broad s), 9.53 (1H, broad s), 8.10-8.03 (1H, m), |
| | | | 7.54 (1H, dd), 7.26 (1H, dt), 6.53 (1H, s), 2.28 (3H, s), |
| | | | 1.32 (9H, s) |
| 21 | 338.77 | 339 | 13.10 (1H, broad s), 9.95 (1H, broad s), 7.69-7.52 (2H, m), |
| | | | 7.33-7.24 (1H, m), 6.69 (1H, s), 4.02-3.91 (1H, m), 2.54 (3H, |
| | | | s), 1.23 (6H, d) |
| 22 | 378.83 | 379 | 9.95 (1H, s), 8.20 (1H, d), 7.70 (1H, dd), 7.61 (1H, dd), |
| | | | 7.30 (1H, t), 6.69 (1H, s), 3.66-3.58 (1H, m), 2.54 (3H, s), |
| | | | 1.88-1.20 (10H, m) |
| 23 | 394.40 | 395 | 10.37 (1H, s), 8.20 (1H, d), 8.00 (2H, d), 7.76 (2H, d), |
| | | | 6.75 (1H, s), 3.66-3.58 (1H, m), 2.55 (3H, s), 2.00-1.00 (10H, m) |
| 24 | 396.41 | 397 | 10.31 (1H, s), 8.57 (1H, t), 7.98 (2H, d), 7.78 (2H, d), |
| | | | 6.76 (1H, s), 2.56 (3H, s), 1.59 (2H, quint.), 1.38-1.22 (8H, m), |
| | | | 0.88 (3H, t) |
| 25 | 354.33 | 355 | 10.36 (1H, s), 8.20 (1H, d), 7.99 (2H, d), 7.77 (2H, d), |
| | | | 6.76 (1H, s), 4.05-3.95 (1H, m), 2.57 (3H, s), 1.28 (6H, d) |
| 26 | 380.85 | 381 | 9.83 (1H, s), 8.54 (1H, t), 7.75 (1H, dd), 7.61 (1H, dd), |
| | | | 7.30 (1H, dt), 6.72 (1H, s), 3.30-3.23 (2H, m), 2.55 (3H, s), |
| | | | 1.57 (2H, m), 1.38-1.24 (8H, m), 0.87 (3H, t) |
| 27 | 419.20 | 420 | 10.36 (1H, s), 8.83 (1H, t), 7.97 (2H, d), 7.78 (2H, d), |
| | | | 6.80 (1H, s), 3.71 (2H, q), 3.67 (2H, t), 2.58 (3H, s) |
| 28 | 324.74 | 325 | 9.83 (1H, s), 8.56 (1H, t), 7.73 (1H, dd), 7.60 (1H, dd), |
| | | | 7.30 (1H, dt), 6.72 (1H, s), 3.32 (2H, quint.), 2.56 (3H, s), |
| | | | 1.18 (3H, t) |
| 29 | 400.84 | 401 | 9.80 (1H, s), 8.65 (1H, t), 7.77 (1H, dd), 7.59 (1H, dd), |
| | | | 7.35-7.18 (6H, m), 6.73 (1H, s), 3.51 (2H, q), 2.90 (2H, t), |
| | | | 2.55 (3H, s) |
| 30 | 406.86 | 407 | 9.78 (1H, s), 8.71 (1H, t), 7.78 (1H, dd), 7.60 (1H, dd), |
| | | | 7.35 (1H, dd), 7.31 (1H, dt), 6.98-6.94 (2H, m), 6.74 (1H, s), |
| | | | 3.53 (2H, q), 3.12 (2H, t), 2.56 (3H, s) |
| 31 | 382.78 | 383 | 9.82 (1H, s), 8.93 (1H, t), 7.78 (1H, dd), 7.59 (1H, dd), |
| | | | 7.31 (1H, dt), 6.78 (1H, s), 4.17 (2H, q), 4.09 (2H, d), 2.56 (3H, s), |
| | | | 1.23 (3H, t) |
| 32 | 513.96 | 514 | 9.88 (1H, s), 8.34 (1H, d), 7.69 (1H, dd), 7.61 (1H, dd), |
| | | | 7.36 (5H, m), 7.31 (1H, dt), 6.72 (1H, s), 5.08 (2H, s), |
| | | | 4.02-1.23 (9H, m), 2.55 (3H, s) |
| 33 | 336.75 | 337 | 9.88 (s, 1H), 8.75 (t, 1H), 7.75 (dd, 1H), 7.60 (dd, 1H), |
| | | | 7.28 (dt, 1H), 6.73 (s, 1H), 6.0-5.85 (m, 1H), 5.2 (dd, 2H), 3.95 (t, |
| | | | 2H), 2.55 (s, 3H) |
| 34 | 379.82 | 380 | 9.62 (bs, 1H), 7.96 (dd, 1H), 7.58 (dd, 1H), 7.28 (dt, 1H), |
| | | | 6.74 (s, 1H), 3.63 (bs, 2H), 3.39 (bs, 2H), 2.50 (bs, 4H), |
| | | | 2.39 (s, 3H), 2.22 (s, 3H). |
| 35 | 393.82 | 394 | 9.83 (1H, s), 8.54 (1H, s), 8.08 (2H, d), 7.84 (1H, t), |
| | | | 7.77-7.70 (3H, m), 7.55 (1H, dd), 7.26 (1H, dt), 2.21 (3H, s) |
| 36 | 461.82 | 462 | 9.89 (1H, s), 8.53 (1H, s), 8.41-8.37 (2H, m), 8.25 (1H, d), |
| | | | 7.98 (1H, t), 7.74 (1H, dd), 7.57 (1H, dd), 7.26 (1H, dt), |
| | | | 2.21 (3H, s) |
| 37 | 472.72 | 474 | 9.85 (1H, s), 8.47 (1H, s), 8.00 (2H, d), 7.94 (2H, d), |
| | | | 7.74 (1H, dd), 7.57 (1H, dd), 7.26 (1H, dt), 2.21 (3H, s) |
| 38 | 407.85 | 408 | 9.81 (1H, s), 8.43 (1H, d), 7.96 (2H, d), 7.75 (1H, dd), |
| | | | 7.56 (1H, dd), 7.51 (2H, d), 7.26 (1H, dt), 2.41 (3H, s), 2.20 (3H, |
| | | | s) |
| 39 | 423.85 | 424 | 9.78 (1H, s), 8.41 (1H, s), 8.00 (2H, d), 7.77 (1H, dd), |
| | | | 7.57 (1H, dd), 7.26 (1H, dt), 7.22 (2H, d), 3.87 (3H, s), 2.21 (3H, |
| | | | s) |
| 40 | 465.93 | 466 | 9.81 (1H, s), 8.42 (1H, s), 7.99 (2H, d), 7.77 (1H, dd), |
| | | | 7.57 (1H, dd), 7.26 (1H, dt), 7.20 (2H, d), 4.09 (2H, t), 2.21 (3H, |
| | | | s), 1.70 (2H, quint.), 1.41 (2H, sext.), 0.91 (3H, t) |
| 41 | 453.87 | 454 | 9.84 (1H, s), 8.42 (1H, s), 7.77 (1H, dd), 7.68 (1H, dd), |
| | | | 7.57 (1H, dd), 7.30-7.21 (2H, m), 3.86 (6H, s), 2.21 (3H, s) |
| 42 | 428.26 | 428 | 9.84 (1H, s), 8.47 (1H, s), 8.09 (2H, d), 7.79 (2H, d), |
| | | | 7.74 (1H, dd), 7.56 (1H, dd), 7.26 (1H, dt), 2.21 (3H, s) |
| 43 | 437.87 | 438 | 9.81 (1H, s), 8.03 (2H, d), 7.77 (1H, dd), 7.57 (1H, dd), |
| | | | 7.27 (1H, dt), 7.18 (2H, d), 6.79 (1H, s), 4.15 (2H, q), 2.57 (3H, s), |
| | | | 1.34 (3H, t) |
| 44 | 477.82 | 478 | 9.86 (1H, s), 8.49 (1H, d), 8.23 (2H, d), 7.77-7.70 (3H, m), |
| | | | 7.57 (1H, dd), 7.26 (1H, dt), 2.21 (3H, s) |
| 45 | 461.82 | 462 | 9.88 (1H, s), 8.53 (1H, d), 8.30 (2H, d), 8.10 (2H, d), |
| | | | 7.73 (2H, d), 7.73 (1H, dd), 7.56 (1H, dd), 7.26 (1H, dt), 2.22 (3H, |
| | | | s) |
| 46 | 411.81 | 412 | 9.84 (1H, s), 8.46 (1H, s), 8.17 (2H, dd), 7.75 (1H, dd), |
| | | | 7.56 (3H, m), 7.26 (1H, dt), 2.21 (3H, s). |
| 47 | 397.81 | 398 | 9.75 (1H, s), 8.34 (2H, s), 7.91 (1H, s), 7.78 (1H, dd), |
| | | | 7.55 (1H, dd), 7.26 (1H, dt), 3.73 (3H, s), 2.23 (3H, s) |
| 48 | 418.83 | 419 | 9.90 (1H, s), 8.51 (1H, s), 8.25 (2H, d), 8.19 (2H, d), |
| | | | 7.72 (1H, dd), 7.56 (1H, dd), 7.26 (1H, dt), 2.21 (3H, s) |
| 49 | 472.72 | 474 | 9.86 (1H, s), 8.02 (2H, d), 7.92 (2H, d), 7.74 (1H, dd), |
| | | | 7.57 (1H, dd), 7.27 (1H, dt), 6.83 (1H, s), 2.58 (3H, s) |
| 50 | 465.93 | 466 | 9.83 (1H, s), 8.03 (2H, d), 7.77 (1H, dd), 7.58 (1H, dd), |
| | | | 7.27 (1H, dt), 7.19 (2H, d), 6.79 (1H, s), 4.09 (2H, t), 2.57 (3H, s), |
| | | | 1.71 (2H, quint.), 1.41 (2H, sext.), 0.92 (3H, t) |
| 51 | 423.85 | 424 | 9.84 (1H, s), 8.42 (1H, s), 7.77 (1H, dd), 7.68 (1H, dd), |
| | | | 7.57 (1H, dd), 7.30-7.21 (2H, m), 3.86 (6H, s), 2.21 (3H, s) |
| 52 | 477.82 | 478 | 9.87 (1H, s), 8.25 (2H, d), 7.75 (1H, dd), 7.70 (2H, d), |
| | | | 7.58 (1H, dd), 7.27 (1H, dt), 6.84 (1H, s), 2.60 (3H, s) |
| 53 | 407.85 | 408 | 9.84 (1H, s), 7.98 (2H, d), 7.76 (1H, dd), 7.57 (1H, dd), |
| | | | 7.51 (2H, d), 7.27 (1H, dt), 6.80 (1H, s), 2.57 (3H, s), 2.42 (3H, s) |
| 54 | 461.82 | 462 | 9.91 (1H, s), 8.43 (1H, d), 8.37 (1H, s), 8.25 (1H, d), |
| | | | 7.97 (1H, t), 7.73 (1H, dd), 7.58 (1H, dd), 7.28 (1H, dt), 6.84 (1H, |
| | | | s), 2.62 (3H, s) |
| 55 | 393.82 | 394 | 9.87 (1H, s), 8.10 (2H, d), 8.84 (1H, t), 7.71-7.70 (3H, m), |
| | | | 7.57 (1H, dd), 7.27 (1H, dt), 6.82 (1H, s), 2.58 (3H, s) |
| 56 | 428.26 | 428 | 9.86 (1H, s), 8.11 (2H, d), 7.81-7.73 (3H, m), 7.57 (1H, dd), |
| | | | 7.27 (1H, dt), 6.83 (1H, s), 2.59 (3H, s) |
| 57 | 437.87 | 438 | 9.81 (1H, s), 8.03 (2H, d), 7.77 (1H, dd), 7.57 (1H, dd), |
| | | | 7.27 (1H, dt), 7.18 (2H, d), 6.79 (1H, s), 4.15 (2H, q), 2.57 (3H, s), |
| | | | 1.34 (3H, t) |
| 58 | 453.87 | 454 | 9.87 (1H, s), 7.76 (1H, dd), 7.71 (1H, dd), 7.57 (1H, dd), |
| | | | 7.48 (14H, d), 7.27 (1H, dt), 7.22 (1H, d), 6.78 (1H, s), 3.87 (3H, |
| | | | s), 3.86 (3H, s), 2.57 (3H, s) |
| 59 | 461.82 | 462 | 9.88 (1H, s), 8.32 (2H, d), 8.10 (2H, d), 7.73 (1H, dd), |
| | | | 7.57 (1H, dd), 7.27 (1H, dt), 6.86 (1H, s), 2.60 (3H, s) |
| 60 | 397.81 | 398 | 9.80 (1H, s), 8.33 (1H, s), 7.91 (1H, s), 7.75 (1H, dd), |
| | | | 7.56 (1H, dd), 7.27 (1H, dt), 6.80 (1H, s), 3.73 (3H, s), 2.63 (3H, s) |
| 61 | 340.80 | 341 | d 10.35 (broad s, 1H), 9.84 (s, 1H), 8.53 (s, 1H), 7.74 (dd, |
| | | | 1H), 7.60 (dd, 1H), 7.28 (td, 1H), 3.72 (q, 2H), 2.25 (s, 3H), |
| | | | 1.26 (t, 3H) |
| 62 | 368.86 | 369 | 10.33 (broad t, 1H), 9.85 (s, 1H), 8.54 (s, 1H), 7.77 (dd, 1H), |
| | | | 7.61 (dd, 1H), 7.31 (td, 1H), 3.70 (q, 2H), 2.26 (s, 3H), |
| | | | 1.68 (quint., 2H), 1.38 (sext., 2H), 0.93 (t, 3H) |
| 63 | 416.90 | 417 | 10.45 (1H, broad t), 9.81 (1H, s), 8.55 (1H, s), 7.79 (1H, dd), |
| | | | 7.62 (1H, dd), 7.32-7.22 (6H, m), 3.93 (2H, q), 3.02 (2H, t), |
| | | | 2.26 (3H, s) |
| 64 | 340.80 | 341 | 9.83 (s, 1H), 8.55 (t, 1H), 7.73 (dd, 1H), 7.60 (dd, 1H), |
| | | | 7.30 (td, 1H), 6.71 (s, 1H), 3.3 (2H under water), 2.55 (s, 3H), |
| | | | 1.17 (t, 3H) |
| 65 | 394.81 | 395 | 13.22 (s, 1H), 9.51 (s, 1H), 8.52-8.50 (m, 1H), 8.16 (dd, 1H) |
| | | | 7.78 (td, 1H), 7.72 (s, 1H), 7.56 (dd, 1H), 7.46 (d, 1H), |
| | | | 7.33-7.23 (m, 2H), 2.26 (s, 3H) |
| 66 | 414.24 | 414 | 10.09 (1H, s), 8.12 (2H, d), 7.90 (1H, t), 7.76 (2H, t), |
| | | | 7.69 (1H, dd), 7.57 (1H, dd), 7.29 (1H, dt), 7.22 (1H, s) |
| 67 | 505.69 | 506 | 9.67 (s, 1H), 8.25 (dd, 1H), 8.18 (dd, 1H), 7.77 (td, 2H), |
| | | | 7.56-7.50 (m, 2H), 7.35 (s, 1H), 7.26 (dt, 1H) |
| 68 | 433.33 | 433 | 9.71 (1H, s), 8.66 (1H, t), 8.24 (1H, s), 7.83 (1H, dd), |
| | | | 7.61 (1H, dd), 7.31 (1H, dt), 3.71 (2H, t), 3.50 (2H, q), 2.75 (2H, |
| | | | t), 2.68 (2H, t), 2.26 (3H, s), 1.99 (2H, quint.) |

### Example 69

Title compounds of the Examples were tested in the biological test described above and were found to exhibit an IC₅₀ of below 10 μM. For example, the following representative compounds of the examples exhibited the following IC₅₀ values:

### Example 4

3.71 μM

### Example 17

8.51 μM

### Example 26

0.40 μM

### Example 30

0.71 μM

### Example 31

0.63 μM

### Example 32

0.66 μM

### Example 33

5.60 μM

### Example 50

8.33 μM

### Example 51

5.87 μM

1. A compound of formula I, 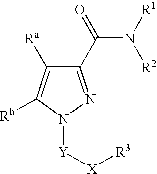
wherein
either
R¹ represents an aryl group or a heteroaryl group, both of which are optionally substituted by one or more substituents selected from G¹ and B¹, which B¹ group may itself be further substituted by one or more substituents selected from G², Z (wherein Z is not directly attached to an aryl or a heteroaryl group) and B² (which B² group is optionally further substituted by one or more substituents selected from G³, B³ and Z, wherein Z is not attached to an aryl or a heteroaryl group); and
R² represents H or C₁₋₈ alkyl, which latter group is optionally substituted by one or more halo groups;
or
when R² represents C₁₋₈ alkyl optionally substituted by halo, R¹ and R² may be linked together forming a further 5- to 7-membered ring, optionally containing 1 to 3 heteroatoms and/or 1 to 3 double bonds, which ring is itself optionally substituted by one or more substituents selected from G¹, Z (when the ring is not aromatic in nature) and B¹ (which B¹ group is optionally substituted as described above);
R³ represents C₁₋₈ alkyl, heterocycloalkyl, aryl or heteroaryl, all of which groups are optionally substituted by one or more substituents selected from G¹, Z (when Z is not directly attached to an aryl or a heteroaryl group) and B¹ (which B¹ group is optionally substituted as described above);
X represents a direct bond or —N(R^{4a})—;
Y represents —C(O)—, —C(S)— or —S(O)₂—;
B¹, B² and B³ independently represent, on each occasion when used above, C₁₋₈ alkyl, heterocycloalkyl, aryl or heteroaryl;
G¹, G² and G³ independently represent, on each occasion when used above, halo, cyano, —N₃, —NO₂, —ONO₂ or -A¹-R^{4b};
wherein A¹ represents a spacer group selected from —C(Z)A²-, —N(R⁵)A³-, —OA⁴-, —S— or —S(O)ₙA⁵-, in which:
A² represents a single bond, —O—, —S— or —N(R⁵)—;
A³ represents A⁶, —C(Z)N(R⁵)C(Z)N(R⁵)—, —C(Z)N(R⁵)C(Z)O—, —C(Z)N(R⁵)S(O)ₙN(R⁵)—, —C(Z)S—, —S(O)ₙ—, —S(O)ₙN(R⁵)C(Z)N(R⁵)—, —S(O)ₙN(R⁵)C(Z)O—, —S(O)ₙN(R⁵)S(O)ₙN(R⁵)—, —C(Z)O—, —S(O)ₙN(R⁵)— or —S(O)ₙO—;
A⁴ represents A⁶, —S(O)ₙ—, —C(Z)O—, —S(O)ₙN(R⁵)— or —S(O)ₙO—;
A⁵ represents a single bond, —N(R⁵)— or —O—;
A⁶ represents a single bond, —C(Z)- or —C(Z)N(R⁵)—;
Z represents, on each occasion when used above, a substituent connected by a double bond, which is selected from ═O, ═S, ═NR^{4b}, ═NN(R^{4b})(R⁵), ═NOR^{4b}, ═NS(O)₂N(R^{4b}(R⁵), ═NCN, ═CHNO₂ and ═C)R^{4b}(R⁵);
R^{4a} represents, on each occasion when used above, H, C₁₋₈ alkyl or a heterocycloalkyl group, which latter two groups are optionally substituted by one or more substituents selected from G⁴, Q and B⁵ (which B⁵ group is optionally substituted by one or more substituents selected from G⁵, Q (when Q is not directly attached to an aryl or a heteroaryl group) and B⁵.
R^{4b} and R⁵ independently represent, on each occasion when used above, H or B⁴, which B⁴ group is itself optionally substituted by one or more substituents selected from G⁴, Q (when Q is not directly attached to an aryl or a heteroaryl group) and B⁵ (which B⁵ group is itself optionally substituted as described above); or
when R^{4b} and/or R⁵ represent optionally substituted B⁴ groups, then any pair thereof may, for example when present on the same atom or on adjacent atoms, be linked together to form, with those, or other relevant, atoms, a 5- to 7-membered ring, optionally containing 1 to 3 heteroatoms and/or 1 to 3 double bonds, which ring is itself optionally substituted by one or more substituents selected from G⁶, Q (when the ring is not aromatic in nature) and B⁴ (which B⁴ group is optionally substituted as described above);
B⁴, B⁵ and B⁶ independently represent on each occasion when used above C₁₋₈ alkyl, heterocycloalkyl, aryl or heteroaryl;
G⁴, G⁵ and G⁶ independently represent on each occasion when used above, halo, cyano, N₃, —NO₂, —ONO₂ or -A⁷-R⁶;
wherein A⁷ represents a spacer group selected from —C(Q)A⁸-, —N(R⁵)A⁹-, —OA¹⁰-, —S— or —S(O)ₙA¹¹-, in which:
A⁸ represents a single bond, —O—, —S— or —N(R⁷)—;
A⁹ represents A¹², —C(Q)S—, —S(O)ₙ—, —C(Q)O—, —S(O)ₙN(R⁷)—, —S(O)ₙO—, —C(Q)N(R⁷)C(Q)N(R⁷)—, —C(Q)N(R⁷)C(Q)O—, —C(Q)N(R⁷)S(O)ₙN(R⁷)—, —S(O)ₙN(R⁷)C(Q)N(R⁷)—, —S(O)ₙN(R⁷)C(Q)O— or —S(O)ₙN(R⁷)S(O)ₙN(R⁷)—;
A¹⁰ represents A¹², —S(O)ₙ—, —C(Q)O—, —S(O)ₙN(R⁷)— or —S(O)ₙO—;
A¹¹ represents a single bond, —N(R⁷)— or —O—;
A¹² represents a single bond, —C(Q)- or —C(Q)N(R⁷)—;
Q represents, on each occasion when used above, a substituent connected by a double bond, which is selected from ═O, ═S, ═NR⁶, ═NN(R⁶)(R⁷), ═NOR⁶, ═NS(O)₂N(R⁶(R⁷), ═NCN, ═CHNO₂ and ═C)R⁶(R⁷);
R⁶ and R⁷ independently represent, on each occasion when used above, H, C₁₋₈ alkyl, heterocycloalkyl, aryl or heteroaryl, which latter four groups are optionally substituted by one or more groups selected from halo, C₁₋₆ alkyl (optionally substituted by one or more halo groups), —N(R⁸)R⁹, —OR⁸, —ON)₂ and —SR⁸; or
when they do not represent H, any pair of R⁶ and R⁷ may, for example when present on the same atom or on adjacent atoms, be linked together to form, with those, or other relevant, atoms, a 5- to 7-membered ring, optionally containing 1 to 3 heteroatoms and/or 1 to 3 double bonds, which ring is itself optionally substituted by one or more groups selected from halo, C₁₋₈ alkyl (optionally substituted by one or more halo groups), —N(R³)R⁰, —OR⁸, —ONO₂ and —SR⁸;
R⁸ and R⁹ independently represent, on each occasion when used above, H or C₁₋₆ alkyl, which latter group is optionally substituted by one or more halo groups;
n represents, on each occasion when used above, 1 or 2; and
R^{a} and R^{b} independently represents H, halo or C₁₋₆ alkyl (which alkyl group is optionally substituted by one or more halo or C₁₋₆ alkoxy groups (which alkoxy group may itself be substituted by one or more halo group)), wherein at least one of R^{a} and R^{b} does not represent H,
or a pharmaceutically-acceptable salt thereof,
provided that, when R² and R⁴ both represent H, Y represents —C(O)—, R^{b} represents methyl and;
(i) X represents a direct bond and R³ represents methyl, then R¹ does not represent 2,6-dimethylphenyl or 2-chloro-6-methylphenyl; and
(ii) X represents —N(R^{4a})— in which R^{4a} represents H and R¹ represents 4-[(2-aminosulfonyl)phenyl]phenyl, then R¹ does not represent 5-bromo-2-pyridyl.

2. A compound as claimed in claim 1, wherein R¹ represents aryl or heteroaryl, both of which are optionally substituted by one or more groups selected from B¹ and G¹.

3. A compound as claimed in claim 1 or claim 2, wherein R² represents H.

4. A compound as claimed in claim 1, wherein R³ represents C₁₋₈ alkyl, heterocycloalkyl, aryl or heteroaryl, all of which are optionally substituted by one or two groups selected from B¹ and G¹.

5. A compound as claimed in claim 1, wherein R^{4a} represents H.

6. A compound as claimed in claim 1, wherein R^{a} and R^{b} independently represent H, C₁₋₄ alkyl or halo.

7. A compound as claimed in claim 1, wherein B¹ represents C₁₋₃ alkyl, aryl or heteroaryl, all of which are optionally substituted by one or more G² groups.

8. A compound as claimed in claim 1, wherein G¹ represents halo, cyano or -A¹R^{4b}.

9. A compound as claimed in claim 1, wherein G² represents halo.

10. A compound as claimed in claim 1, wherein A¹ represents —S—, —C(Z)_A²-, —OA⁴- or —S(O)₂A⁵.

11. A compound as claimed in claim 1, wherein A² represents —O—.

12. A compound as claimed in claim 1, wherein A⁴ and A⁵ independently represent a single bond.

13. A compound as claimed in claim 1, wherein Z represents ═O.

14. A compound as claimed in claim 1, wherein R^{4b} represents B⁴.

15. A compound as claimed in claim 1, wherein B⁴ represents C₁₋₄ alkyl or aryl, both of which groups are optionally substituted by one or more groups selected from G⁴ and B⁵.

16. A compound as claimed in claim 1, wherein G⁴ represents halo.

17. A compound as claimed in claim 1, wherein B⁵ represents aryl.

18. A compound as claimed in claim 1, wherein R¹ represents optionally substituted phenyl, quinolinyl, pyridyl, isoquinolinyl, 1,3-benzodioxolyl or 1,4-benzodioxanyl.

19. A compound as claimed in claim 1, wherein R¹ is optionally substituted by one or more substitutions selected from halo or C₁₋₃ alkyl, which alkyl group is linear or branched and/or is optionally substituted by one or more halo group.

20. A compound as claimed in claim 1, wherein R³ represents optionally substituted C₁₋₆ alkyl, C₅₋₆ cycloalkyl, C₂₋₄ alkenyl, piperidinyl, piperazinyl, phenyl pyridyl, imidazolyl or morpholinyl.

21. A compound as claimed in claim 1, wherein R³ is optionally substituted by one or more substituents selected from halo, cyano, C₁₋₆ alkyl (which alkyl group is linear or branched and/or is optionally substituted by one or more halo group), phenyl, thienyl, pyridyl, oxazolyl, thiazolyl, ═O, —OR¹⁰, —C(O)OR¹¹, —SR¹² and —S(O)₂R¹³ (wherein R¹⁰, R¹¹ and R¹² independently represent C₁₋₆ alkyl, which alkyl group is optionally substituted by one or more halo atoms or aryl groups and R¹³ represents phenyl, optionally substituted by one or more fluoro atoms).

22. (canceled)

23. A pharmaceutical formulation including a compound of formula I, as defined in claim 1, or a pharmaceutically acceptable salt thereof, in admixture with a pharmaceutically acceptable adjuvant, diluent or carrier.

24. A method which comprises administering a compound of formula I, as defined in claim 1 but without the provisos, or a pharmaceutically acceptable salt thereof, for the manufacture of a for the treatment of a disease in which inhibition of the activity of a lipoxygenase is desired and/or required.

25. A method as claimed in claim 24 wherein the lipoxygenase is 15-lipoxygenase.

26. A method as claimed in claim 24, wherein the disease is inflammation and/or has an inflammatory component.

27. A method as claimed in claim 26 wherein the inflammatory disease is asthma, chronic obstructive pulmonary disease, pulmonary fibrosis, an allergic disorder, rhinitis, inflammatory bowel disease, an ulcer, inflammatory pain, fever, atherosclerosis, coronary artery disease, vasculitis, pancreatitis, arthritis, osteoarthritis, rheumatoid arthritis, conjunctivitis, iritis, scleritis, uveitis, a wound, dermatitis, eczema, psoriasis, stroke, diabetes, autoimmune diseases, Alzheimer's disease, multiple sclerosis, sarcoidosis, Hodgkin's disease or another malignancy.

28. A method of treatment of a disease in which inhibition of the activity of a lipoxygenase is desired and/or required, which method comprises administration of a therapeutically effective amount of a compound of formula I as defined in claim 1 but without provisos, or a pharmaceutically-acceptable salt thereof, to a patient suffering from, or susceptible to, such a condition.

29. A combination product comprising:
(A) a compound of formula I as defined in claim 1 but without the provisos, or a pharmaceutically-acceptable salt thereof; and
(B) another therapeutic agent that is useful in the treatment of inflammation, wherein each of components (A) and (B) is formulated in admixture with a pharmaceutically-acceptable adjuvant, diluent or carrier.

30. A combination product as claimed in claim 29 which comprises a pharmaceutical formulation including a compound of formula I as defined in claim 1 but without the provisos, or a pharmaceutically-acceptable salt thereof; another therapeutic agent that is useful in the treatment of inflammation, and a pharmaceutically-acceptable adjuvant, diluent or carrier.

31. A combination product as claimed in claim 29 which comprises a kit of parts comprising components:
(a) a pharmaceutical formulation including a compound of formula I as defined in claim 1 but without the provisos, or a pharmaceutically-acceptable salt thereof; in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier, which components (a) and (b) are each provided in a form that is suitable for administration in conjunction with the other.

32. A process for the preparation of a compound of formula I as defined in claim 1, which comprises:
(i) for compounds of formula I in which R³ represents a tertiary C₁₋₈ alkyl, tertiary heterocycloalkyl, aryl or heteroaryl group and R^{b} represents C₁₋₆ alkyl, optionally substituted as defined in claim 1, or halo, reaction of a compound corresponding to a compound of formula I in which R^{b} represents hydrogen, with an appropriate base, followed by:
(a) for compounds of formula I in which R^{b} represents an optionally submitted C₁₋₆ alkyl group, quenching with an electrophile of formula II,
R^{c}L^{1a} II
wherein R^{c} represents C₁₋₆ alkyl, which group is optionally substituted by one or more halo or methoxy groups and L^{1a} represents a suitable leaving group; or
(b) for compounds of formula I in which R^{b} represents halo, quenching with an electrophile that provides a source of halide ions;
(ii) for compounds of formula I in which, when Y is —S(O)₂— and X is —N(R^{4a})— in which R^{4a} is B⁴, reaction of a compound of formula III, 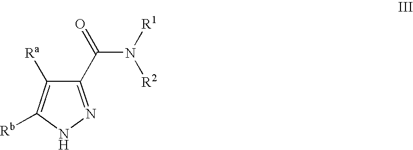
wherein R^{a}, R^{b}, R¹ and R² are as defined in claim 1, with a compound of formula IV,
R³—X^{a}—Y-L¹ IV
wherein, when Y represents —S(O)₂—, X^{a} represents a direct bond or ═N(B⁴)— or, for all other values of Y, X^{a} represents X as defined in claim 1, L¹ represents a suitable leaving group and R³ and Y are as defined in claim 1;
(iii) for compounds of formula I in which X represents a single bond and Y represents —C(O)—, reaction of a compound of formula III as defined above with a compound of formula V,
R³C(O)OH V
wherein R³ is as defined in claim 1;
(iv) for compounds of formula I in which R³ represents a primary or secondary C₁₋₈ alkyl or a secondary heterocycloalkyl group, X represents a direct bond and Y represents a —C(O)— or a —C(S)— group, reaction of a compound of formula III as defined above with a compound of formula VI,
R³═Y^{a} VI
wherein Y^{a} represents —C(O)— or —C(S)— and R³ is as defined in claim 1;
(v) for compounds of formula I, in which X represents —NH— and Y represents —C(O)— or —C(S)—, reaction of a compound of formula III as defined above with a compound of formula VII,
R³N═Y^{a} VII
wherein R³ is as defined in claim 1 and Y^{a} is as defined above;
(vi) for compounds of formula I in which Y represents —C)O— or —C(S)—, reaction of a compound of formula III as defined above with:
(a) a compound of formula VIII,
Cl—Y^{a}—Cl VIII
(b) a compound of formula IX, 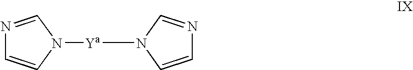
wherein, in both cases, Y^{a} is as defined above; or
(c) when Y represents —C(O)—, triphosgene, followed by:
(1) for compounds of formula I in which X represents a direct bond, reaction with a compound of formula X,
R³M X
wherein M represents a metal, or a salt or complex thereof, and R³ is as defined in claim 1; or
(2) for compounds of formula I wherein X represents:
(I) —N)R^{4a})—, reaction with an amine of formula XI,
R³N(H)R^{4a} XI
wherein R³ and R^{4a} are as defined in claim 1; or
(II) a direct bond and R³ represents a nitrogen-containing heterocycloalkyl group, in which a nitrogen atom of the heterocycloalkyl group is attached directly to the Y substituent of the compound of formula I, reaction with a corresponding secondary amine of the nitrogen-containing heterocycloalkyl group;
(vii) for compounds of formula I in which X represents —N(R^{4a})— and R^{4a} is other than hydrogen, reaction of a corresponding compound of formula I in which X represents —N(H)— with a compound of formula XII,
R^{4c}-L¹ XIII
wherein R^{4c} represents any value of R^{4a} as defined in claim 1 other than H and L′ us as defined above;
(viii) for compounds of formula I in which Y represents —C(S)—, reaction of a corresponding compound of formula I in which Y represents —C(O)— with a suitable reagent for the conversion of a carbonyl group to a thiocarbonyl group;
(ix) reaction of a compound of formula XIII, 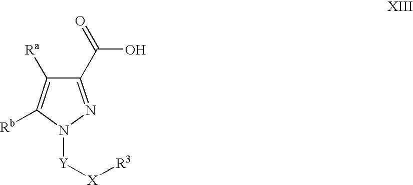
wherein R^{a}, R^{b}, R³, Y and X are as defined in claim 1, with a compound of formula XIV,
HN(R¹)(R²) XIV
wherein R¹ and R² are as defined in claim 1;
(x) reaction of a compound of formula XV, 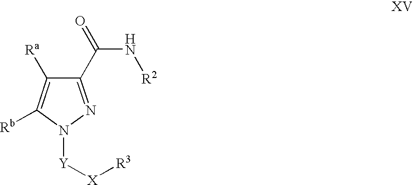
wherein R^{a}, R^{b}, R², R³, Y and X are as defined in claim 1, with a compound of formula XVI,
R¹-L² XVI
wherein L² represents a suitable leaving group and R¹ is as defined in claim 1; or
(xi) for compounds of formula I in which one of R^{a} or R^{b} represents an optionally substituted C₁₋₆ alkyl group and the other represents H (as appropriate), reaction of a compound corresponding to a compound of formula I in which one of R^{a} and R^{b} represents bromo or iodo and the other represents H with a suitable organolithium base, followed by quenching with a compound of formula II, as defined above.
